(19) United States
(12) Patent Application Publication
(10) Pub. No.: US 2019/0126265 A1
(43) Pub. Date: May 2, 2019

(54) METHOD OF DIVERTING THE FIRST FRACTION OF SAMPLE AWAY FROM A LATERAL FLOW ASSAY

(71) Applicant: Tokitae LLC, Bellevue, WA (US)

(72) Inventors: Bernhard Hans Weigl, Seattle, WA (US); Ozgur Emek Yildirim, Bellevue, WA (US)

(21) Appl. No.: 15/800,170

(22) Filed: Nov. 1, 2017

(51) Int. Cl. B01L 3/00; G01N 33/543; G01N 33/569

(52) U.S. Cl. CPC B01L 3/5023; G01N 33/54386; G01N 33/54306; G01N 33/5695; B01L 2400/086; B01L 2300/0877; B01L 2200/10; B01L 2200/12; B01L 2400/0406

(57) ABSTRACT

A fluidic assay device and related methods of use and manufacture are disclosed. A fluidic assay device includes a diversion structure pad having a fixed bed volume and capable of producing a capillary flow rate to preferentially draw a first volume of sample fluid delivered by a fluid delivery device to a loading region away from an assay flow path. A second volume of sample fluid delivered from the delivery device flows from the loading region into an assay flow path. For example, the assay flow path may include a lateral flow membrane and components for capture and detection of an analyte of interest. The fluidic assay device can be used with, sandwich immunoassays and other assays suited for use in lateral flow format, for example.

If an Application Data Sheet (ADS) has been filed on the filing date of this application, it is incorporated by reference herein. Any applications claimed on the ADS for priority under 35 U.S.C. §§ 119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC § 119(e) for provisional patent applications, for any and all parent, grandparent, great-grandparent, etc. applications of the Priority Application(s)).

### PRIORITY APPLICATIONS

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

In an aspect, a lateral flow assay device includes, but is not limited to, a diversion pad having a fixed bed volume and capable of producing a first capillary flow rate of a sample fluid, a sample pad capable of producing a second capillary flow rate of the sample fluid, a loading region adapted to receive the sample fluid, wherein the loading region is configured for fluid communication with the diversion pad and the sample pad, and a lateral flow membrane downstream of the sample pad and including one or more capture component adapted to capture an analyte of interest in the sample fluid, wherein the first capillary flow rate is greater than the second capillary flow rate. In addition to the foregoing, other device aspects are described in the claims, drawings, and text forming a part of the disclosure set forth herein.

In an aspect, a method of manufacturing a lateral flow assay device includes, but is not limited to, providing a support, disposing a lateral flow membrane layer including at least one capture component specific to an analyte of interest on a support, disposing a sample pad layer on the support, disposing a diversion pad layer on the support adjacent the sample pad layer and separated from the lateral flow membrane layer, and forming a loading structure adapted for fluid communication with the diversion pad layer and the sample pad layer, wherein the diversion pad layer is formed from a material capable of producing a first capillary flow rate of a sample fluid containing the analyte of interest and having a fixed bed volume per volume of material, and wherein the sample pad layer is formed from a material capable of producing a second capillary flow rate of the sample fluid, wherein the first capillary flow rate is greater than the second capillary flow rate. In addition to the foregoing, other method aspects are described in the claims, drawings, and text forming a part of the disclosure set forth herein.

In an aspect, a method of diverting a portion of a sample fluid away from an assay flow path of a lateral flow assay device includes, but is not limited to, receiving a sample fluid at a loading region of a lateral flow assay device from a sample delivery device, wherein the sample delivery device is adapted to deliver in sequence a first portion of the sample fluid followed by a second portion of the sample fluid; preferentially drawing the first portion of the sample fluid from the loading region into a diversion pad having a fixed bed volume until the fixed bed volume has been filled; and after the fixed bed volume has been filled, preferentially drawing the second portion of the sample fluid into a sample pad upstream of the assay flow path of the lateral flow assay device; wherein until the fixed bed volume of the diversion pad is filled, the diversion pad produces a higher capillary flow rate of the sample fluid than does the sample pad to preferentially draw the first portion of the sample fluid into the diversion pad, and wherein after the fixed bed volume has been filled, the sample pad produces a higher capillary flow rate of the sample fluid than does the diversion pad to preferentially draw the second portion of the sample fluid into the sample pad. In addition to the foregoing, other method aspects are described in the claims, drawings, and text forming a part of the disclosure set forth herein.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A, 1B, and 1C depict an example of a lateral flow assay.

FIG. 2A is a top view of a lateral flow assay device.

FIG. 2B is a side view of the lateral flow assay device of FIG. 2A.

FIG. 3A is a simplified side view of a lateral flow assay device as shown in FIGS. 2A and 2B.

FIG. 3B is a simplified top view of a lateral flow assay device as shown in FIGS. 2A and 2B.

FIG. 4A depicts a first stage of loading of a lateral flow assay device.

FIG. 4B depicts a second stage of loading of a lateral flow assay device.

FIG. 5A is top view of an embodiment of a lateral flow assay device.

FIG. 5B is side view of the embodiment of the lateral flow assay device of FIG. 5A.

FIG. 6A is a simplified top view of a lateral flow assay device as shown in FIGS. 5A and 5B.

FIG. 6B depicts a first stage of loading the lateral flow assay device of FIG. 6A.

FIG. 6C depicts a second stage of loading a lateral flow assay device of FIG. 6A.

FIGS. 7A, 7B, and 7C depict variants of loading a lateral flow assay device.

FIG. 8 is a side view of an embodiment lateral flow assay device including a loading pad.

FIG. 9 is a side view of another embodiment lateral flow assay device including a loading pad.

FIG. 10 is a side view of an embodiment of a lateral flow assay device.

FIG. 11 is a top view of an embodiment of a lateral flow assay device.

FIG. 12 is a side view of an embodiment of a lateral flow assay device.

FIG. 13 is a side view of an embodiment of a lateral flow assay device.

FIG. 14A is an illustration of a lateral flow assay device including a housing.

FIG. 14B is a cross-section view of the lateral flow assay device of FIG. 14A.

FIG. 15 is a flow diagram of method of manufacturing a lateral flow assay device.

FIG. 16 is a flow diagram of a method of diverting a portion of a sample fluid away from an assay flow path of a lateral flow assay device.

FIG. 17 depicts a fluidic assay device including a sample diversion structure.

FIG. 18 is a flow diagram of a method of diverting a portion of a sample fluid away from an assay flow path of a fluidic assay device.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

The present invention relates to devices for performing lateral flow assays, and in particular to a lateral flow assay device that includes a portion for diverting a fixed-volume first portion of a sample away from the assay flow path prior to directing the remainder of the sample toward the assay flow path.

Lateral flow assays devices are commonly used to perform assays to detect presence or absence, or in some cases quantity, of an analyte in a sample. For example, lateral flow assays are used to detect hormones indicative of pregnancy or ovulation, infectious disease vectors, and drugs of abuse and various other analytes, e.g., as discussed in U.S. Pat. No. 4,703,017 to Campbell et al.; M. Sajid, A.-N. Kawde, and M. Daud, (2015) “Designs, formats and applications of lateral flow assay: A literature review,” J. Saudi Chem. Soc., Vol. 19, pp. 689-705; and “Rapid Lateral Flow Test Strips: Considerations for Product Development,” Lit. No. TB500EN00EM Rev. C 12/13, © 2013, EMD Millipore Corporation, Billerica, Mass., each of which is incorporated herein by reference. Lateral flow assay devices are generally inexpensive and simple to use. A lateral flow assay device typically includes one or more porous or fibrous materials forming an assay flow path. Dried reagents are stored in the flow path. A sample fluid applied to the lateral flow assay device flows through the assay flow path in response to capillary forces, solubilizing and interacting with dried reagents as it moves though the assay flow path. Ultimately, the analyte of interest binds to a capture reagent immobilized in the assay flow path, resulting in a detectable signal indicating the presence or absence of the analyte. In some lateral flow assay devices, the signal is visually detectable by a human to provide qualitative information regarding presence or absence of the analyte. Some lateral flow assays are sufficiently sensitive that it is possible to obtain quantitative information regarding the amount of analyte in the sample. In some cases, lateral flow assay devices are used in combination with hand-held or table-top readers. Lateral flow assays frequently are used to perform immunochromatographic tests such as sandwich assays or competitive binding assays, e.g. as discussed in U.S. Pat. No. 4,376,110 to David et al. and U.S. Pat. No. 4,855,240 to Rosenstein et al., both of which are incorporated herein by reference.

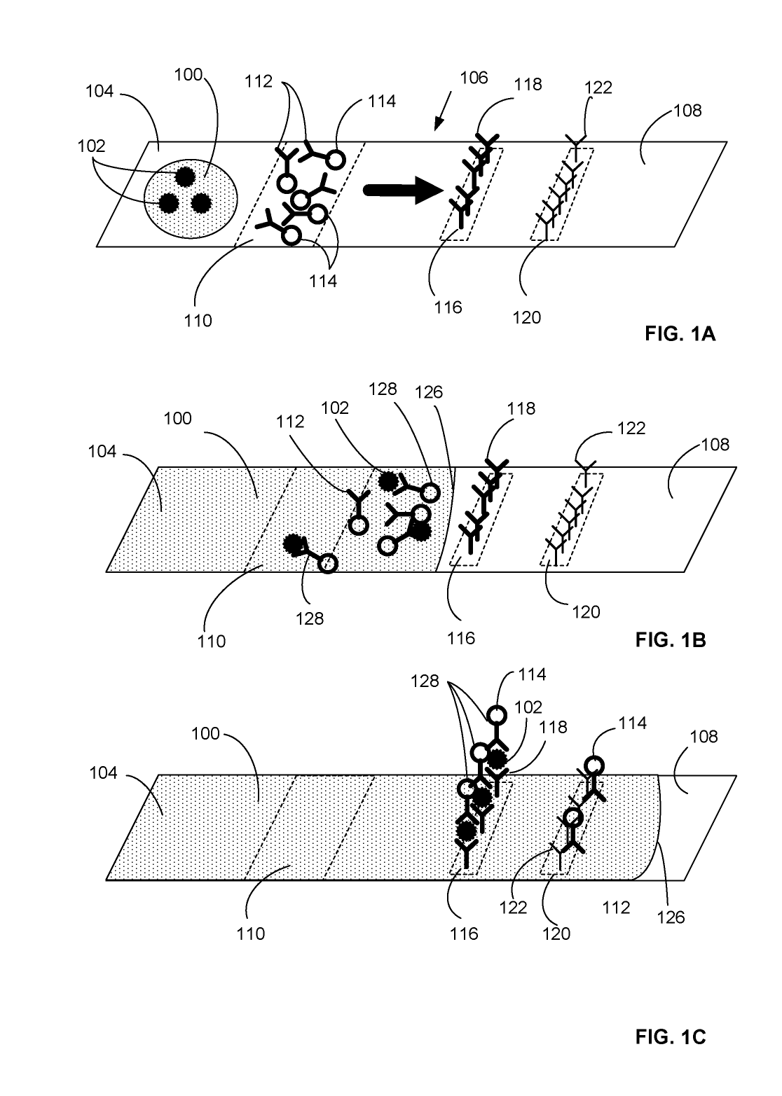

FIGS. 1A, 1B, and 1C illustrate an example of a sandwich assay performed in a lateral flow format. In FIG. 1A, a sample fluid 100 containing analyte of interest 102 is applied at first end 104 of lateral flow assay device 106, and moves via capillary forces to second end 108, in the direction indicated by the heavy black arrow. Lateral flow assay device 106 includes a conjugate region 110 containing conjugate antibodies 112 specific to analyte 102. Conjugate antibodies 112 are conjugated to one or more detectable component 114, which may be, for example, latex beads, colloidal gold particles, other colloidal metals, colloidal carbon, fluorescent or luminescent labels, quantum dots, upconverting phosphores, bioluminescent markers, enzymes, magnetic or paramagnetic particles, dyes, electroactive compounds, or other suitable labels or markers. (Peter Chun, “Chapter 5. Colloidal Gold and Other Labels for Lateral Flow Immunoassays”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference) In an aspect, detectable components are contained within and subsequently released from liposomes. Lateral flow assay device 106 also includes a test line 116, containing antibodies 118 immobilized on the material forming the assay flow path, and a control line 120 containing antibodies 122, which are specific to conjugate antibodies 112.

FIG. 1B depicts lateral flow assay device 106 after sufficient time has passed for sample fluid 100 to spread to fill first end 104 of lateral flow assay device and travel downstream through lateral flow assay device 106, to the location of flow front 126. As sample fluid 100 moves passes conjugate region 110, it solubilizes conjugate antibodies 112 and interacts with them to form bound conjugate antibodies 128, which are bound to analyte 102. Some of conjugate antibodies 112 remain unbound.

FIG. 1C depicts lateral flow assay device 106 after flow front 126 of sample fluid 100 has travelled to second end 108 of lateral flow assay device 106. As sample fluid 100 travels past test line 116, analyte 102 with bound conjugate antibodies 128 is bound by antibodies 118. Unbound conjugate antibodies 112 bind to antibodies 122 at control line 120. Detectable component 114 conjugated to bound conjugate antibodies 128 at test line 116, and unbound conjugate antibodies 112 at control line 120, produces a detectable signal that indicates presence of analyte 102 (at test line 116) and presence of properly functioning assay components (at control line 120), respectively.

Various types of assays can be performed in a lateral flow format. Although a sandwich assay is described herein, it should be understood that other types of assays, using different types of capture components for capturing and visualizing analytes of interest may be used instead, for example competitive binding assays, inhibition assays, or serum assays, e.g., as discussed in U.S. Pat. No. 4,703,017 to Campbell et al.; M. Sajid, A.-N. Kawde, and M. Daud, (2015) “Designs, formats and applications of lateral flow assay: A literature review,” J. Saudi Chem. Soc., Vol. 19, pp. 689-705, which is incorporated herein by reference.

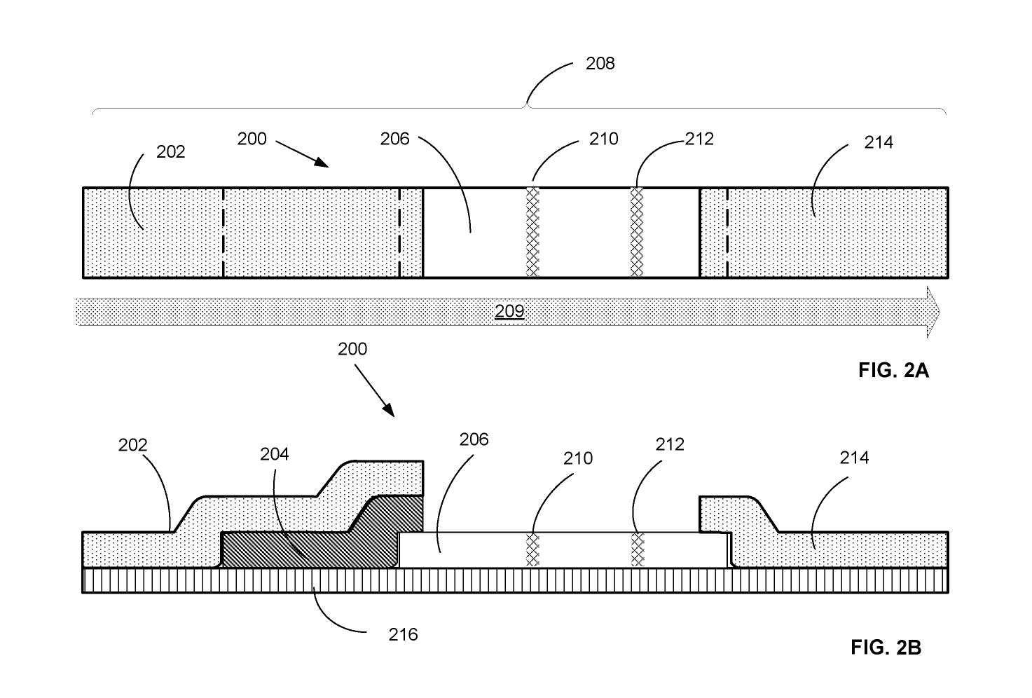

FIG. 2A is a top view of a conventional lateral flow assay device 200. FIG. 2B is a side view of the conventional lateral flow assay device 200 of FIG. 2A. The lateral flow assay device depicted in FIGS. 2A and 2B is used to perform an assay of the type illustrated in FIGS. 1A-1C, for example, or other types of assays. Lateral flow assay device 200 includes sample pad 202, which is in fluid communication with conjugate pad 204. In use, a sample fluid containing an analyte of interest is applied to sample pad 202, which functions to control the rate at which sample fluid enters conjugate pad 204. Sample pad 202 may contain proteins, detergents, viscosity enhancers, buffers, salts, or other materials that improve the properties of the sample fluid.

Conjugate pad 204 contains dried conjugate (e.g., conjugate antibodies and detectable component as discussed in connection with FIGS. 1A-1C). In various aspects, conjugate pad 204 is formed from glass fiber, polyesters, cotton, or rayon, e.g. as discussed in “Rapid Lateral Flow Test Strips: Considerations for Product Development,” Lit. No. TB500EN00EM Rev. C 12/13, © 2013, EMD Millipore Corporation, Billerica, Mass., which is incorporated herein by reference. Lateral flow assay device includes lateral flow membrane 206, a porous membrane with well-defined capillary flow properties that provides a uniform and controlled flow of sample fluid along the assay flow path 208 (in the direction indicated by the shaded arrow 209) to test line 210 and control line 212. Lateral flow membrane materials include nitrocellulose, polyvinylidene fluoride, charge-modified nylon, polyether sulfone, nitrocellulose acetate, glass fiber, cellulose, paper, silica, a porous synthetic polymer, polyester, nylon, cotton, a sintered material, a woven material, or a non-woven material. In an aspect, lateral flow membrane materials are treated with surfactant to improve the wettability of the membrane. (See, e.g. Michael A. Mansfield, “Chapter 6. Nitrocellulose Membranes for Lateral Flow Immunoassays: A Technical Treatise”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3; E. J. Flynn, J. Arndt, L. Brothier, and M. A. Morris (2013), “Control of pore structure formation in cellulose nitrate polymer membranes,” Advances in Chem. Science, Vol. 2, Issue 2, June 203, pp. 9-18; and “Rapid Lateral Flow Test Strips: Considerations for Product Development,” Lit. No. TB500EN00EM Rev. C 12/13, © 2013, EMD Millipore Corporation, Billerica, Mass., each of which is incorporated herein by reference, for discussion of lateral flow membrane and conjugate pad materials).

Test line 210 contains immobilized antibodies specific to the analyte of interest, bound irreversibly to lateral flow membrane 206, and a control line 212 contains immobilized antibodies specific to conjugate antibodies, bound irreversibly to lateral flow membrane 206, as discussed above in connection with FIGS. 1A-1C. Absorbent pad 214 (which may also be referred to as a wick) is located at the downstream end of assay flow path 208 and captures sample fluid from the end of assay flow path 208, thus permitting additional fluid to flow into assay flow path 208 from sample pad 202 and increasing the volume of sample that flows through the assay. In various aspects, absorbent pad 214, which is located downstream of lateral flow membrane 206 and adapted to receive fluid that has passed through the lateral flow membrane. In various aspects, absorbent pad 214 includes at least one of cellulose, high-density cellulose, glass, polyester, nylon, cotton, mono-component fiber, or bi-component fiber (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference).

As shown in FIG. 2B, sample pad 202, conjugate pad 204, lateral flow membrane 206, and absorbent pad 214 are formed on backing 216. In various aspects, backing 216 includes a non-porous plastic film or card, including one or more of polystyrene, vinyl (poly vinyl chloride or PVC), or polyester. In various aspects, the backing includes an adhesive, which may be covered by a release liner. Thickness of the backing may be for example 0.0005 to 0.015 inches, with thicker materials typically used for stand-alone test strips, while thinner materials may be used in a holder or housing (see, e.g. Jennifer S. Ponti, “Chapter 3. Material Platform for the Assembly of Lateral Flow Immunoassay Test Strips”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference). Although it is typical that lateral flow assay devices are formed on a backing, in some cases the materials forming the lateral flow assay device are sufficiently self-supporting that the backing can be omitted.

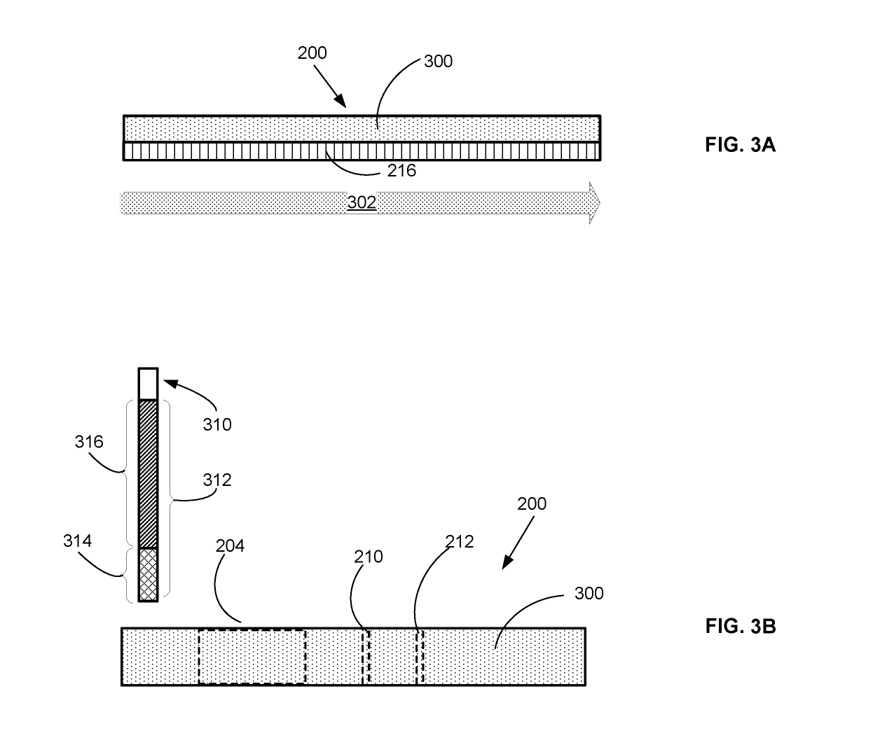

FIG. 3A is a simplified side view of a conventional lateral flow assay device of the type shown in FIGS. 2A and 2B. Lateral flow assay device 200 includes assay flow path 300 (which includes components depicted in FIGS. 3A and 3B, e.g. a sample pad, conjugate pad, lateral flow membrane, and absorbent pad) and backing, however, for simplicity of illustration the individual components of the assay flow path are not depicted in this and other similar figures herein. Assay flow direction is indicated by shaded arrow 302.

FIG. 3B is a simplified top view of a lateral flow assay device as shown in FIGS. 2A and 2B. Assay flow path includes conjugate pad 204, test line 210, and control line 212. Also shown in FIG. 3B is sample delivery device 310, which contains sample fluid 312, which includes a first portion 314 and second portion 316. In practice, sample fluid 312 is not uniform, and the first portion 314 may differ from the second portion 316 with regard to concentration of analyte of interest, presence of contaminants, or other qualities that may influence the result of the assay. For example, if the sample has been preprocessed, e.g. by filtration concentration of a biomarker, the first portion of fluid may not have been properly processed to the same concentration as the second portion of the sample. In another example, the first portion of fluid may be non-representative due to exposure to the atmosphere or other environmental factors that may contaminate or otherwise deteriorate the sample. For example, in an assay for detecting tuberculosis Mycobacterium tuberculosis cell wall antigen lipoarabinomannan (TB LAM) in whole blood, a first portion of the blood sample may be contaminated with LAM from environmental mycobacteria on the skin, which, if not discarded, would result in a false positive result. As another example, a first portion of a urine sample may be contaminated by microorganisms from the skin during voiding/sample collection. In another example, a first portion of blood from a finger stick may be representative of capillary blood but not systemic blood with regard to concentration of analytes affected by oxygen and CO₂, or markers of tissue damage. For example, the first portion of the sample may include interferences due to capillary damage. This is especially relevant with regard to assays for inflammatory or capillary damage-related host response markers such as C-Reactive Protein (CRP), soluble triggering receptor expressed on myeloid cells (sTREM), and related or similar markers, in which it is desirable to distinguish between systemically present markers and markers related to localized capillary damage.

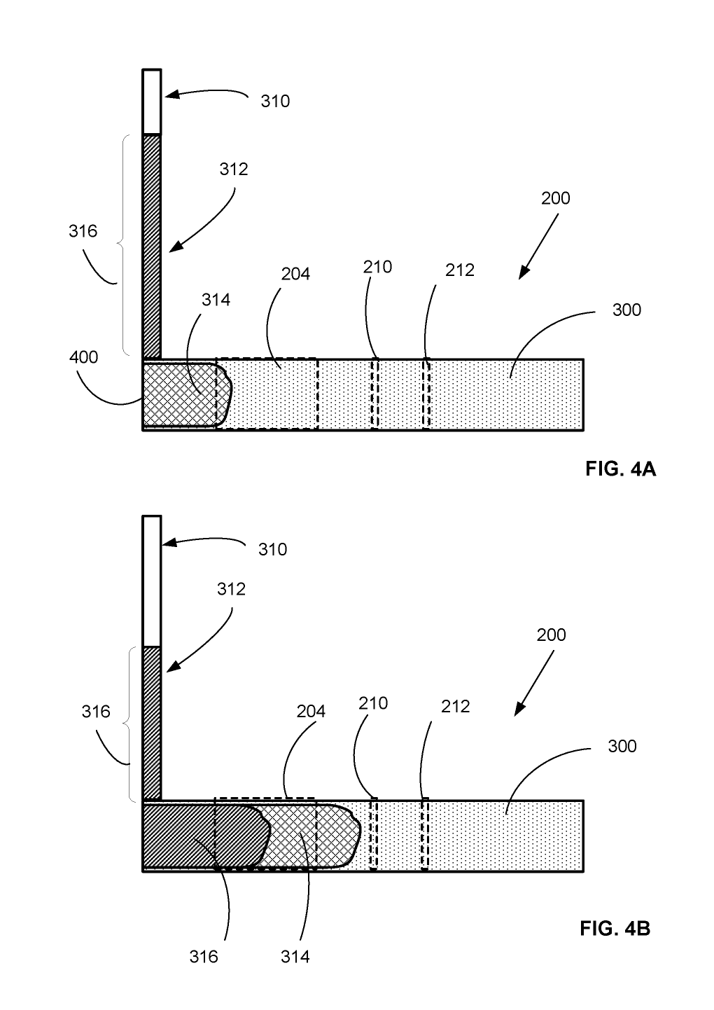

As will be discussed in connection with FIGS. 4A and 4B, the variation between first portion 314 and second portion 316 of sample fluid 312 can lead to inconsistencies in assay results.

It should be noted that in FIG. 3A, FIG. 4A and subsequent simplified illustrations showing loading of a lateral flow assay device with a sample delivery device (e.g., lateral flow assay device 200 and sample delivery device 310, respectively, in FIG. 4A) the lateral flow assay device is depicted in a top view, and the sample delivery device is depicted as lying substantially the same plane as the lateral flow assay device, i.e., in a side view. In practice, the lateral flow assay device is typically held substantially horizontally, with the backing, if present, below and supporting the lateral flow assay device, and the sample delivery device (which may be a pipette or the like) is held perpendicular or angled with respect to the horizontal plane of the lateral flow assay device, such that the flow of sample fluid into the lateral flow assay device is assisted by gravity. Hence, FIG. 4A and similar figures herein are intended to convey information about the relative amounts of sample fluid in the sample delivery device at different stages of loading of the lateral flow assay device, but are not intended to accurately reflected the angle of the sample delivery device with respect to the lateral flow assay device.

FIG. 4A depicts a first stage of loading of a conventional lateral flow assay device 200. Sample delivery device 310 contacts lateral flow assay device 200 at an upstream end 400 of assay flow path 300, and first portion 314 of sample fluid 312 flows onto lateral flow assay device 200, while second portion 316 remains in sample delivery device 310. Conjugate pad 204, test line 210, and control line 212 are as described previously.

FIG. 4B depicts a second stage of loading of a lateral flow assay device. As can be seen, first portion 314 has advanced across conjugate pad 204 and almost to test line 210, while second portion 316 has advanced partially across conjugate pad 204 although some of second portion 316 is still in sample delivery device 310. Because sample fluid typically (and desirably) advances uniformly across assay flow path 300, first portion 314 and second portion 316 of sample fluid 312 arrive at conjugate pad 204, test line 210, and control line 212 at different times, and hence ‘see’ different concentrations of reagents at these locations. Moreover, in an aspect, first portion 314 (which as noted above may contain different concentrations or amounts of analyte or contaminants) is not representative of sample fluid 312 as a whole, yet it flows through the assay first, hence any materials in first portion 314 that interfere with the assay may cause problems with the essay before second portion 316 has even passed conjugate pad 204, test line 210, and control line 212.

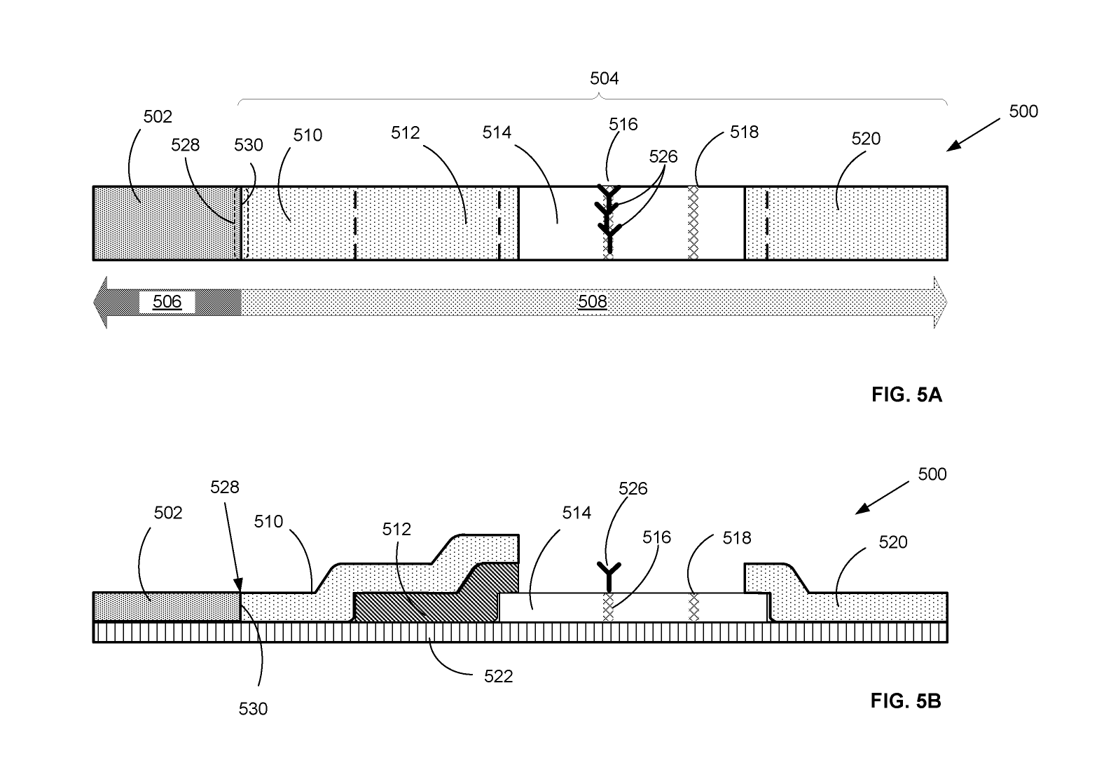

FIGS. 5A and 5B are top and side view, respectively, of a lateral flow assay device 500 that includes a diversion pad 502 that serves to divert a first portion of a sample fluid away from the assay flow path 504 and into diversion pad 502 (in a direction indicated by arrow 506), so that a second portion (expected to be more desirable and/or more representative) is permitted to flow through the assay flow path 504 in the direction indicated by arrow 508. In an aspect, discarding the first portion of the sample allows the results of the assay to be more representative of the sample. This may be particularly useful in quantitative assays, for example.

Lateral flow assay device 500 also includes a number of components that are substantially similar to the components of a conventional lateral flow assay devices, e.g. as described in connection with FIGS. 2A and 3B, including sample pad 510, conjugate pad 512, lateral flow membrane 514, test line 516, control line 518, absorbent pad 520, and backing 522. As noted above, in some cases the materials forming the lateral flow assay device are sufficiently self-supporting that backing 522 can be omitted.

In various aspects, diversion pad 502 is formed from materials similar to those typically used in sample pads in lateral flow assay devices. For example, in an aspect, diversion pad 502 includes at least one of cellulose, glass fiber, cotton, rayon, a woven mesh, and a synthetic non-woven material (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference). For example, suitable materials include, but are not limited to, SureWick® glass fiber pads and cellulose pads from EMD Millipore, Billerica, Mass., and CF1 to CF7 100% cotton linter pads from GE Healthcare Biosciences, Pittsburgh, Pa. As will be discussed herein below, the specific choices of materials for diversion pad 502 and sample pad 510 are important to the functioning of lateral flow assay device 500; specifically, the capillary flow rates of sample fluid produced by diversion pad 502 and sample pad 510 are influenced by the choice of pad materials.

In an aspect, sample pad 510 includes at least one of cellulose, glass fiber, cotton, rayon, a woven mesh, and a synthetic non-woven material (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference). For example, suitable materials include, but are not limited to, SureWick® glass fiber pads and cellulose pads from EMD Millipore, Billerica, Mass. and CF1 to CF7 100% cotton linter pads from GE Healthcare Biosciences, Pittsburgh, Pa.)

Diversion pad 502 has a fixed bed volume and is capable of producing a first capillary flow rate of the sample fluid. Sample pad 510 is capable of producing a second capillary flow rate of the sample fluid, with the first capillary flow rate being greater than the second capillary flow rate.

In substrates having sufficiently small values of capillary or pore diameter, capillary forces are sufficient to overcome gravitational and inertial forces to produce capillary fluid flow. Capillary pressure P_{c} exerted experienced by the liquid in a cylindrical capillary can be expressed by the Young-Laplace equation as:

$${P}_{c} = \frac{2 γ}{r} cos θ \qquad [ EQN . 1 ]$$

where γ is the liquid-air surface tension of the liquid, θ is the contact angle of the liquid on the material forming the capillary, and r is the radius of the capillary. It can be seen that capillary pressure is inversely related to capillary (pore) radius, and is dependent on the properties of the liquid and the material forming the capillary.

The flow of fluid into the porous or fibrous materials, such as those used in the construction of lateral flow assay devices, can be approximated by equations describing capillary flows.

The Washburn equation (see E. W. Washburn, 1921, “The Dynamics of Capillary Flow”, Physical Review, Vo. XVII, No. 3, pp. 273-283, which is incorporated herein by reference) express the distance 1 that a liquid travels into a horizontal capillary in a time t as follows:

$$\begin{matrix}{l}^{2} = ( \frac{γ cos θ}{2 η} ) rt & [ EQN . 2 ] \\ l = \sqrt{\frac{r cos θ}{2}} \sqrt{\frac{γ}{η}} \sqrt{t} & [ EQN . 3 ]\end{matrix}$$

where η is the viscosity of the fluid, and γ, θ, and r are surface tension, contact angle, an capillary radius, respectively, as discussed above. The volume of fluid penetrating into a porous material (e.g., of fluid flowing into a lateral flow membrane) in a time t is proportional to

$$\sqrt{\frac{γ}{η}} \sqrt{t} .$$

The time for fluid to travel a given distance into a capillary is:

$$t = \frac{2 η}{γ} \frac{{l}^{2}}{r cos θ} \qquad [ EQN . 4 ]$$

The rate of flow of a liquid into a horizontal capillary due to capillary pressure can be expressed as:

$$\frac{d l}{d t} = \frac{r}{η} \frac{γ}{4 l} cos θ \qquad [ EQN . 5 ]$$

It should also be noted that as distance l increases, the capillary flow rate eventually decreases to zero.

In an aspect, capillary flow rate can be approximated as being proportional to capillary (or pore) radius, thus, higher capillary flow is obtained with larger capillary or pore size. It should be noted that the Washburn equation does not perfectly describe fluid flow into capillaries, since it neglects the effect of inertia of the fluid, which results in

$$\frac{d l}{d t}$$

being undefined for l=0. In addition, fluid flow in porous media is only approximated by equations describing fluid flow in capillaries, due to the more complex geometries of the openings in porous media relative to the cylindrical openings in capillaries. By including additional terms, fluid inertia can be taken into account (see, e.g., Schoelkopf, J., Gane, P. A. C., Ridgway, C. J., & Matthews, G. P. (2000). “Influence of inertia on liquid absorption into paper coating structures.” Nordic Pulp & Paper Research Journal, 15(5), 422-430., which is incorporated herein by reference). Lastly, the Washburn equation and the interpretation discussed above is only valid for pore sizes that are much smaller than the capillary length scale, λ_{c}, where capillary forces dominate over gravitational forces:

$${λ}_{c} = \sqrt{\frac{γ}{ρ g}} \qquad [ EQN . 6 ]$$

In EQN. 6, γ is surface tension, ρ is density, and g is the gravitational acceleration. This can also be captured by looking at the dimensionless gravitational Bond number

$${B}_{0} = \frac{ρ g {L}^{2}}{γ} \qquad [ EQN . 7 ]$$

L is the characteristic length, and, again, γ is surface tension, ρ is density, and g is gravitational acceleration. When Bo<<1, capillary forces dominate over gravity. Capillary length scale is approximately 2.7 mm for water, thus this condition is easily satisfied for effective pore sizes in lateral flow assay devices.

In practice, materials used in lateral flow assay devices are typically characterized by capillary flow time (the time t for a fluid to travel a specified distance l within the material, see EQN. 4) rather than capillary flow rate

$$\frac{d l}{d t}$$

(see EQN. 5), since the capillary flow rate varies along the length of a material as fluid flows into it, decreasing inversely proportionally to the distance of the flow front from the origin) and is hence difficult to measure. See, e.g. (“Rapid Lateral Flow Test Strips: Considerations for Product Development,” Lit. No. TB500EN00EM Rev. C 12/13, © 2013, EMD Millipore Corporation, Billerica, Mass.), which is incorporated herein by reference. However, the higher the capillary flow rate, the lower the capillary flow time by definition. The viscosity (η) and surface tension (γ) depend on the sample fluid, and the contact angle (θ) depends on both the sample fluid and the capillary material. Thus, materials for lateral flow assays produce a particular capillary flow rate or flow time for a particular sample fluid, and different flow rate and flow time will be obtained with different fluids. Commercially available materials for used in lateral flow assay devices are characterized by capillary flow time for water.

In some aspects, the sample fluid used in assay devices described herein is a biological fluid, for example, a blood sample, amniotic fluid, bile, cerebrospinal fluid, peritoneal fluid, pleural fluid, saliva, seminal fluid, synovial fluid, tears, sweat, vaginal secretion, breast milk, or urine. In some aspects, the sample fluid includes a solvent in which a biological material is dissolved. In an aspect, the solvent is a polar solvent. In an aspect, the solvent is aqueous. In an aspect, at least a portion of the biological material forms a suspension or emulsion in the sample fluid (e.g., a fecal sample may be diluted in a liquid prior to testing in a lateral flow assay device).

As noted above, diversion pad 502 is capable of producing a first capillary flow rate of the sample fluid and sample pad 510 is capable of producing a second capillary flow rate of the sample fluid, with the first capillary flow rate higher than the second capillary flow rate, such that the fluid flows preferentially into the diversion pad until it is filled, before flowing into the sample pad. It is contemplated that the dimensions of the diversion pad are such that fluid flows into and fills the bed volume of the diversion pad before the capillary flow rate of fluid into the diversion pad has decreased to be equal to the initial capillary flow rate of fluid into the sample pad. Hence, although the capillary flow rate of fluid into the diversion pad will decrease as the fluid flows into the diversion pad, the capillary flow rate of fluid into the diversion pad will be higher than the capillary flow rate of fluid into the sample pad until the diversion pad is filled, such that fluid flows preferentially into the diversion pad until it is filled, and subsequently flows into the sample pad.

As can be seen from the equations above, the capillary flow rate depends on pore size and material properties of the pad, as well as distance travelled into the pad by the fluid. Thus, different relative capillary flow rates of diversion pad 502 and sample pad 510 can be obtained by using pads formed from the same material (e.g., cellulose fiber) but having different pore sizes, or by pads having the same pore size but formed from different materials. In some aspects, different materials are used to form the structure of diversion pad 502 and sample pad 510, and in some aspects, the same material is used to form the structure of the pads, but different types or quantities of surface treatments (including, e.g., plasma treatment to increase surface energy or addition of surfactants such as detergents) are performed to diversion pad 502 and sample pad 510 to increase the capillary flow rate of diversion pad 502 relative to sample pad 510.

Although dimensions of the lateral flow assay device 500 and other lateral flow assay devices described herein may vary depending on the particular assay being performed, and are not limited to any specific dimensions, in an aspect the dimensions of the lateral flow assay devices are substantially similar to conventional lateral flow assay devices, e.g. with assay flow path ranging between about 3 mm and about 1 cm wide, about 4 cm and about 10 cm long, and about 100 μm to about 3 mm thick. In an aspect, the thickness of different portions of lateral flow assay device 500 differs depending on the thicknesses of the different pads and membranes and the number of overlapping layers at a given location in the device. In an aspect, diversion pad 502 has a length of between about 2 mm and about 2 cm, which is in addition to the length of the assay flow path.

The viscosity, contact angle, and surface tension of the sample fluid that will be used in the assay should also be taken into account. Specifications for lateral flow assay membrane, sample pad, conjugate pad, and absorbent pad materials typically assume an air atmosphere, but it will be appreciated that surface tension and contact angle are dependent on the gaseous atmosphere in which the assay is performed, as well as the pad material and sample fluid, so if the assay is performed in a non-air atmosphere this must also be taken into account.

Lateral flow membrane 514 is located downstream of sample pad 510 and includes one or more capture component (e.g., an antibody 526 at test line 516) adapted to capture an analyte of interest in a sample fluid applied to the lateral flow assay device. It will be appreciated that various types of capture components and various assay designs can be used in connection with lateral flow assay device 500. Various types of assays and capture components may be used, as discussed herein above, e.g. in connection with FIGS. 1A-1C. In some aspects, capture components and binding components are optimized for performing a quantitative assay for an analyte of interest. In various aspects, binding and/or capture components are adapted for binding markers related to inflammation, tissue damage, or blood vessel damage. In some aspects, binding and/or capture components are adapted for binding C-Reactive Protein or soluble triggering receptor expressed on myeloid cells (sTREM), for example. In some aspects, binding and/or capture components are adapted for binding markers affected by oxygen concentration, carbon dioxide concentration, or pH. In some aspects, the binding and/or capture components are adapted to bind a marker related to sepsis, e.g. a marker related to at least one of enteric bacteria, mycobacteria, or coliform bacteria. In aspects, binding and/or capture components are adapted to bind tuberculosis Mycobacterium tuberculosis cell wall antigen lipoarabinomannan (TB-LAM). Other applications in which quantitative assays are of relevance are quantitative nucleic acid detection, e.g. for HIV-1 RNA (see, e.g., Rohrman, G. A., Leautaud, V., Molyneux, Richards-Kortum, R. R. (2012), “A Lateral Flow Assay for Quantitative Detection of Amplified HIV-1 RNA,” PLoS ONE 7(9); e45611. Doi:10.1371/journal.pone.0045611, which is incorporated herein by reference).

Lateral flow assay device 500 includes a loading region 528 adapted to receive the sample fluid. Loading region 528 is configured for fluid communication with diversion pad 502 and sample pad 510. In an aspect, diversion pad 502 and the sample pad 510 abut each other at a junction 530. In an aspect, loading region 528 includes the junction 530 between the diversion pad and the sample pad.

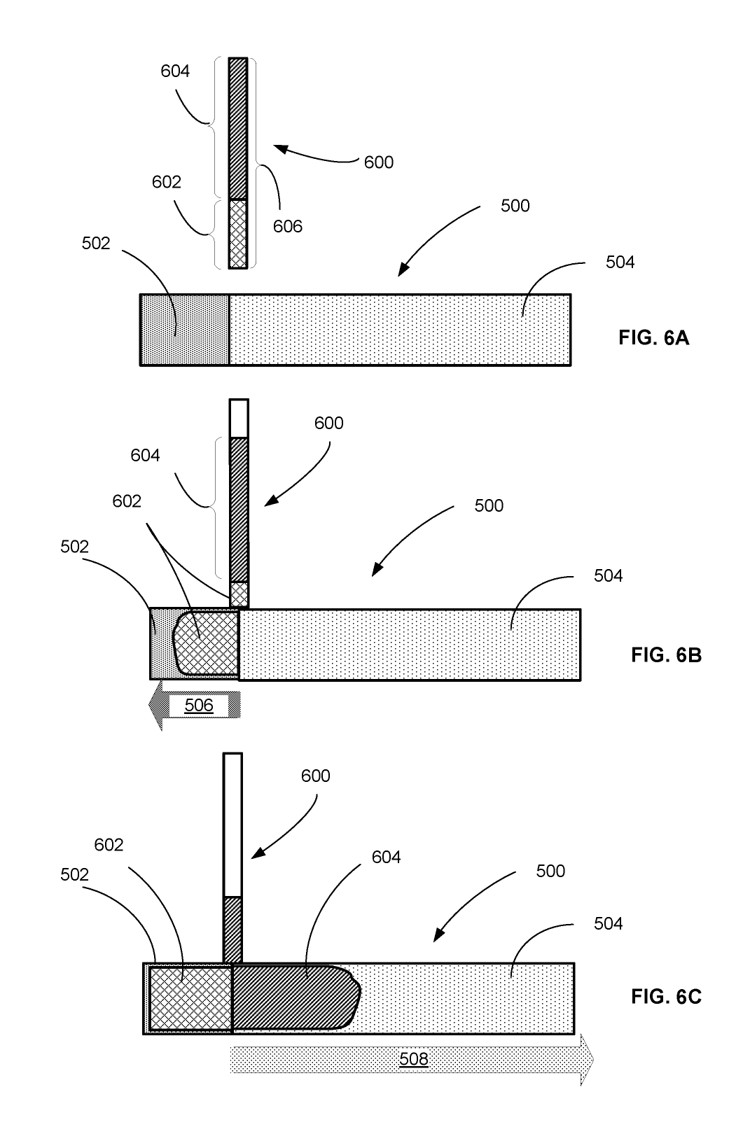

FIG. 6A is a simplified top view of a lateral flow assay device 500 as shown in FIGS. 5A and 5B. Diversion pad 502 and assay flow path 504 are as described above. Sample delivery device 600 is used to deliver a first portion 602 and second portion 604 of a sample fluid 606 to lateral flow assay device 500.

FIG. 6B depicts a first stage of loading the lateral flow assay device of FIG. 6A in which first portion 602 has begun to flow into diversion pad 502, in the direction of arrow 506. In FIG. 6B, a fraction of first portion 602 remains in sample delivery device 600.

FIG. 6C depicts a second stage of loading a lateral flow assay device of FIG. 6A., in which all of first portion 602 has flowed into diversion pad 502, and second portion 604 has begun to flow into assay flow path 504, in the direction indicated by arrow 508. It can be seen that the bed volume (fluid capacity) of diversion pad 502 is equal to the volume of first portion 602. In general, the bed volume is dependent on the total volume of the pad and the porosity of the pad material (the porosity is the amount of air in the 3D structure of the material, expressed as a percent of the total volume of the 3D structure).

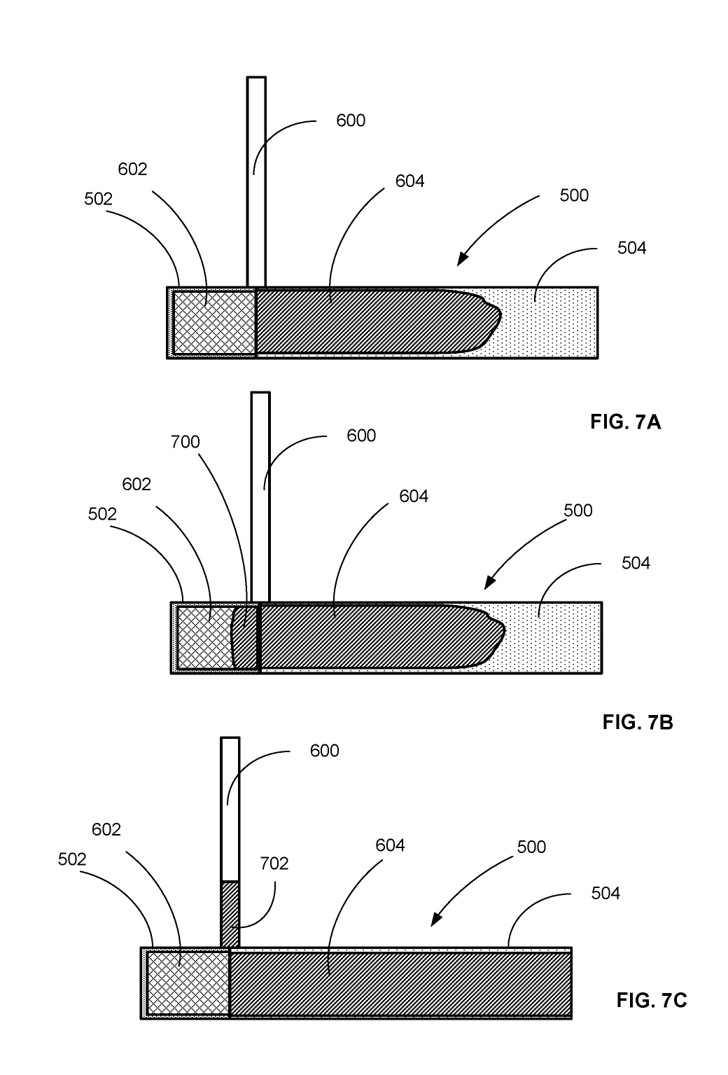

FIGS. 7A, 7B, and 7C depict variants of loading a lateral flow assay device 500 as shown in FIGS. 5A and 5B. Diversion pad 502, assay flow path 504, and sample delivery device 600 are as described above. FIGS. 7A, 7B, and 7C illustrate the effect of different volumes of first and second portions of sample fluid relative to the bed volumes of the diversion pad and assay flow path. FIG. 7A depicts the scenario in which the volume of first portion 602 exactly fills diversion pad 502, but the volume of second portion 604 does not completely fill assay flow path 504. Providing that the volume of second portion 604 passing through the test and control lines (not shown) is sufficient for the assay to function properly, it is not necessary that the assay flow path be completely filled. FIG. 7B depicts the scenario in which the volume of first portion 602 is not sufficient to fill diversion pad 502, so a quantity 700 of second portion 604 flows into diversion pad 502 until it is filled, following which the remainder of second portion 604 flows into assay flow path 504. This assures that only the second portion flows through the assay flow path. However, if the quantity of the second portion that flows through the assay flow path is not sufficient for the assay to function properly, it may be undesirable to have a part of the second portion diverted into the diversion pad. FIG. 7C depicts the scenario in which the volume of first portion 602 exactly fills diversion pad 502, and the volume of second portion 604 is larger than the volume of assay flow path 504. In this case, there is potential for excess fluid 7012 to be released from sample delivery device 600 to overflow from lateral flow assay device 500. An additional scenario is that the volume of first portion 602 is larger than the volume of diversion pad 502, such that after diversion pad 502 has filled, a quantity of the first portion 602 will flow down the assay flow path 504 in advance of second portion 604, comparable to the sample flow depicted in FIGS. 4A and 4B. In order to prevent the first portion 602 of sample from entering the assay flow path 504, it is necessary to use a diversion pad 502 that has a bed volume equal to or greater than the volume of the first portion 602 of the sample. In addition, it is desirable that the volume of the second portion 604 is sufficient for proper functioning of the assay without overflowing of the lateral flow assay device 500. Accordingly, in an aspect, diversion pad 502 is designed to have a bed volume sufficient to accommodate the expected volume of the first portion 602. In an aspect, additional absorbent elements can be added to the lateral flow assay device to capture overflow fluid, for example as described in U.S. Published Patent Application 2013/0309760 to Raj et al., which is incorporated herein by reference. In an aspect, in designing the lateral flow assay, the expected amount of sample fluid available, and expected relative amounts of first portion 602 and second portion 604 in the sample fluid should be taken into account, and the bed volumes of assay device components and/or sample volumes should be adjusted to ensure appropriate fluid volumes in each portion of the lateral flow assay device 500.

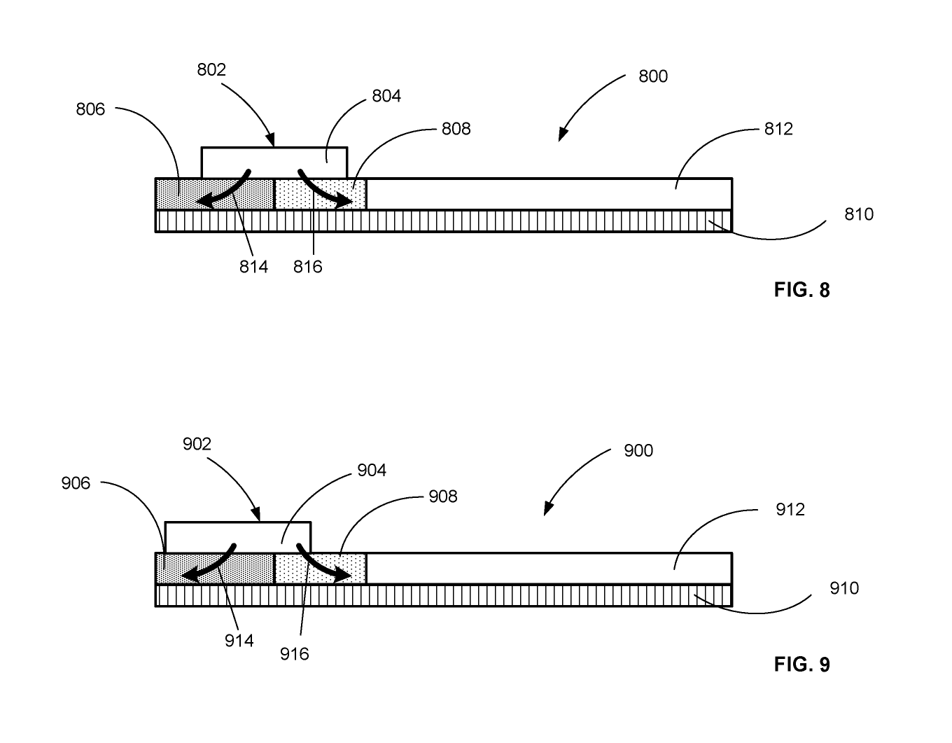

In another aspect, as depicted in FIG. 8, a lateral flow assay device 800 includes a loading region 802 that includes a loading pad 804 positioned above the junction of diversion pad 806 and sample pad 808, such that loading pad 804 overlaps and is in fluidic communication with both diversion pad 806 and the sample pad 808. Diversion pad 806 and sample pad 808 are supported by a backing 810. The remainder of the assay flow path (i.e., the portion downstream of sample pad 808) is indicated at 812, and is generally as described herein above. Loading pad 804 is formed from cellulose, high-density cellulose, glass, polyester, nylon, cotton, mono-component fiber, or bi-component fiber, e.g. as used in absorbent pad 520. In general, loading pad 804 is formed from a material that rapidly takes up sample fluid applied to loading pad 804, and allows it to flow readily to diversion pad 806 and sample pad 808. In use, sample fluid applied to loading region 802 enters loading pad 804, flows preferentially into diversion pad 806, as indicated by arrow 814, until diversion pad 806 is filled, and then into sample pad 808, as indicated by arrow 816. From sample pad 808, sample fluid flows downstream into the remainder 812 of the assay flow path. In an aspect, the capillary flow rate of loading pad 804 is higher than that of either diversion pad 806 or sample pad 808. In an aspect, the fluid capacity (bed volume) of loading pad 804 is sufficient to contain fluid sample applied to loading pad 804 while sample flows into diversion pad 806 and then sample pad 808 and the remainder 812 of assay flow path.

A similar lateral flow assay device 900 is depicted in FIG. 9. Lateral flow assay device 900 includes a loading region 902 that includes a loading pad 904 positioned asymmetrically over diversion pad 906 and sample pad 908, with greater overlap of the diversion pad 906 than the sample pad 908. Lateral flow assay device 900 also includes backing 910. Loading pad 904 overlaps and is in fluid communication with both diversion pad 906 and the sample pad 908, but the asymmetrical positioning favors the flow of sample fluid into diversion pad 906. In use, sample fluid applied to loading region 902 enters loading pad 904, flows preferentially into diversion pad 902, as indicated by arrow 914, until diversion pad 906 is filled, and then into sample pad 908, as indicated by arrow 916. From sample pad 908, sample fluid flows into the remainder 912 of the assay flow path.

As discussed herein above, diversion pad material is selected that provides a higher capillary flow rate than does the sample pad material. However, in some aspects, while sample fluid flows rapidly into diversion pad, the sample fluid may not be retained within the diversion pad and may flow out of the diversion pad and into the sample pad. Therefore, in some aspects, additional materials are added to discourage flow of fluid from the diversion pad to the sample pad and/or encourage retention of fluid in the diversion pad. In one aspect, the diversion pad includes a dispersed hydrogel within the material of the diversion pad, which serves to absorb and retain sample fluid that has been drawn into the sample pad. The dispersed hydrogel may be a superabsorbent polymer of the type used in disposable diapers, for example, such as polyvinyl alcohol, polyacrylamide, or polyacrylate. In other aspects, materials or structures that block or limit fluid flow are placed between the diversion pad and sample pad, as discussed herein below.

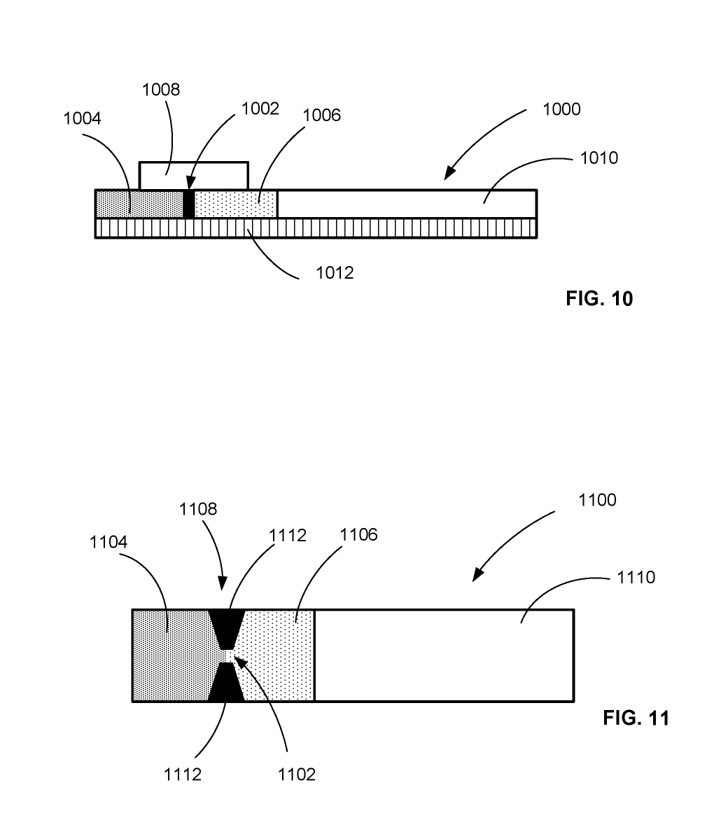

As shown in FIG. 10, in an aspect a lateral flow assay device 1000 includes a fluid impermeable barrier 1002 between diversion pad 1004 and sample pad 1006. Lateral flow assay device 1000 also includes loading pad 1008, the remainder 1010 of the assay flow path, and backing 1012, as described generally herein above. Fluid impermeable barrier 1002 can be formed for example, by an oil, a wax, a plastic or other polymer, a metal, a glass, or a ceramic, for example. Fluid impermeable barrier 1002 blocks the flow of fluid from sample diversion pad 1004 to sample pad 1006 (and vice versa) such that sample fluid enters sample pad 1006 only via loading pad 1008.

In an aspect, rather than using a fluid impermeable barrier between diversion pad 1004 and sample pad 1006, a flow limiting structure can be placed between diversion pad 1004 and sample pad 1006. For example, in an aspect, the flow limiting structure includes a dam formed from a dissolvable material, wherein the dissolvable material is dissolvable by the sample fluid. In another aspect, a flow limiting structure includes a hydrophobic region (e.g., a region at the boundary between the sample and diversion pads is treated to make it sufficiently hydrophobic to limit the flow of (aqueous or polar) sample fluid. In some aspects, flow of non-polar sample fluid can be limited by including a hydrophilic region between the sample and diversion pads.

FIG. 11, is a top view of a lateral flow assay device 1100 that includes a pinch point 1102 between diversion pad 1104 and sample pad 1106, at loading region 1108. Pinch point 1102 functions as a flow limiting structure, as described generally herein above. Lateral flow assay device 1100 may also include a loading pad (not shown). The remainder 1110 of the assay flow path, is as described generally herein above. Pinch point 1102 can be formed for example, by a narrowing in a fluid flow path between diversion pad 1104 and sample pad 1106. In an aspect, the regions 1112 (shown as dark areas in FIG. 11) are barrier regions in diversion pad 1104 and sample pad 1106, formed by an oil, a wax, a plastic or other polymer, a metal, a glass, or a ceramic, for example. In another aspect, regions 1112 are air gaps formed by omitting portions of diversion pad 1104 and/or sample pad 1106 within regions 1112. Pinch point 1102 limits the flow of fluid from sample diversion pad 1104 to sample pad 1106.

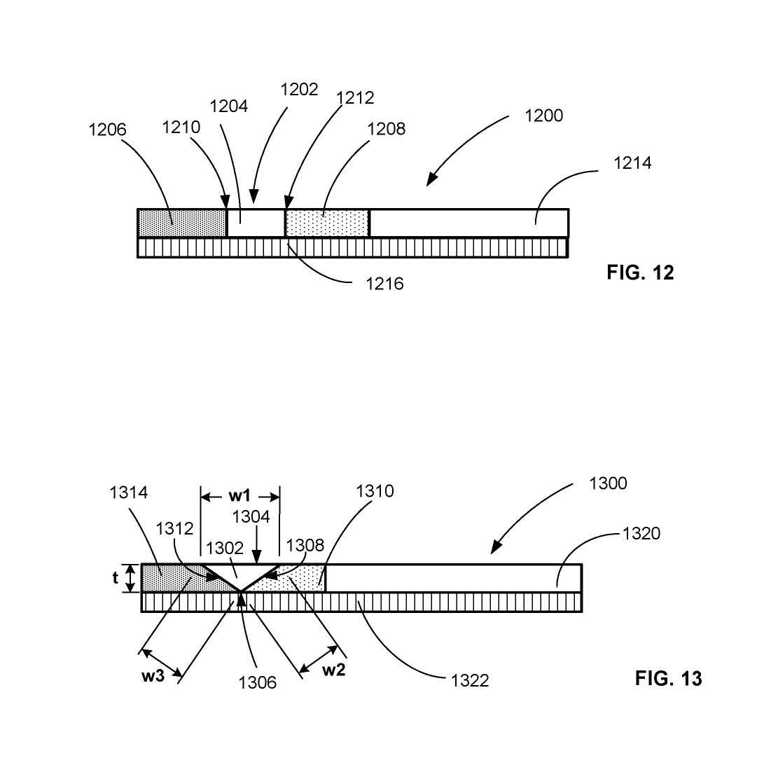

In another aspect, as shown in FIG. 12, a lateral flow assay device 1200 includes a loading region 1202, which includes a loading pad 1204 located between diversion pad 1206 and sample pad 1208, with loading pad 1204 abutting diversion pad 1206 on a first side 1210 of loading pad 1204 and abutting sample pad 1208 on second side 1212 of loading pad 1204. Lateral flow assay device 1200 also includes the remainder 1214 of the assay flow path, and backing 1216, as described generally herein above.

FIG. 13 depicts a lateral flow assay device 1300 having a further variant of a loading pad. In the example of FIG. 13, loading pad 1302 is wedge-shaped, and wider at a top surface 1304 (having a width w1) and narrower at a bottom region 1306 (here shown as narrowing to substantially zero width). Surface 1308, which abuts the sample pad 1310, and the surface 1312, which abuts diversion pad 1314 are angled with respect to the top surface 1304 of the loading pad 1302. Surface 1308 has a width w2 and surface 1312 has a width w3. In the example of FIG. 13, both width w2 and w3 are wider than the thickness t of the loading pad 1302. In other related embodiments, a wedge-shaped loading pad may have just one of the surfaces of the loading pad abutting the diversion pad and the sample pad wider than the thickness of the loading pad. Lateral flow assay device 1300 also includes the remainder 1320 of the assay flow path, and backing 1322, as described generally herein above.

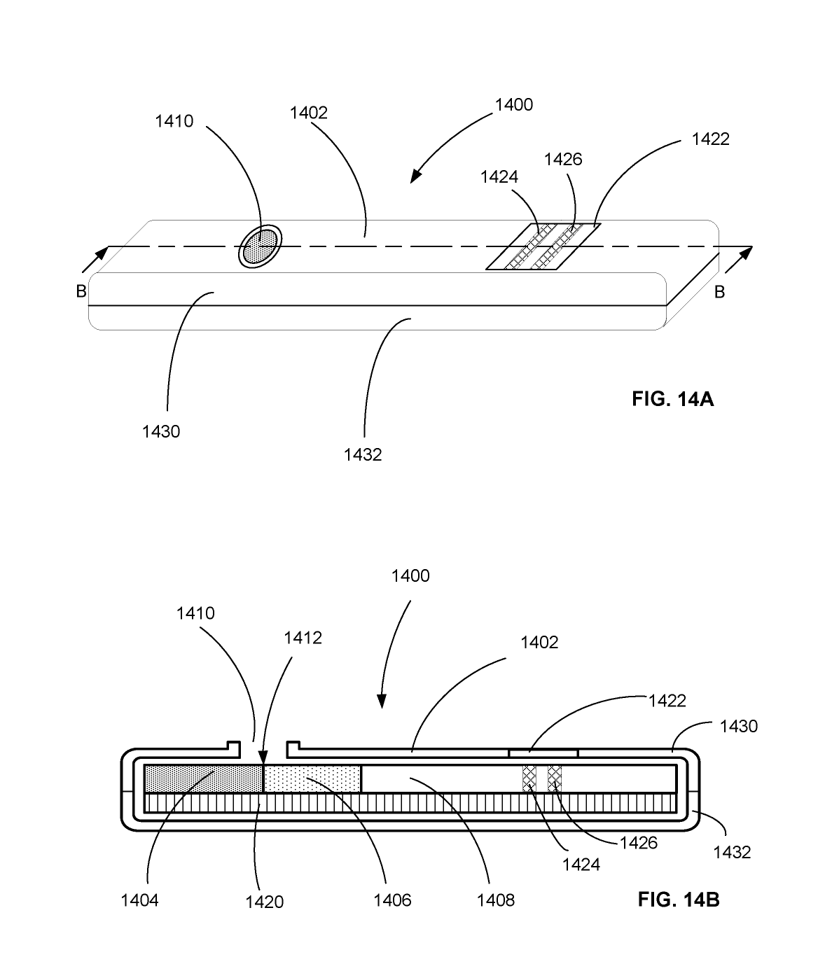

In some aspects, a lateral flow assay device 1400 includes a housing 1402, as depicted in FIGS. 14A and 14B. FIG. 14A is a perspective view of lateral flow assay device 1400, and FIG. 14B is a cross-sectional view taken along section line B-B in FIG. 14A. Housing 1402 is configured (e.g., has sufficient size and shape) to contain the diversion pad 1404, sample pad 1406, and remainder 1408 of the assay flow path (including, e.g. the lateral flow membrane, and other components as described herein above). Housing 1402 includes an access port 1410 in the housing 1402, which is configured (e.g. by location, size, and shape) to permit delivery of fluid to loading region 1412. While housing 1402 and access port 1410 can be constructed with various shapes and dimensions, and are not limited to any particular dimensions, as an example, in some aspects, housing 1402 is from about 5 cm to about 10 centimeters long, about 1 cm to about 2 cm wide, and about 0.5 cm to about 1 cm high. In an aspect, access port 1410 is between about 1 mm and about lcm across, and may be substantially oval as depicted in FIG. 14A, substantially circular, or may have some other shape. In an aspect, access port 1410 is substantially wider at the top than at the bottom, such that it is wide enough at the top to receive the tip of a pipette or other delivery device, but narrow enough at the bottom to prevent the tip from contacting and potentially damaging loading region 1412 of lateral flow assay device 1400. In an aspect, lateral flow assay device 1400 includes a backing 1420 on which the diversion pad 1404, sample pad 1406, and remainder 1408 of the assay flow path (including the lateral flow membrane) are supported. In an aspect, a window 1422 in housing 1402 allows a user to view a test line 1424 and control line 1426. In an aspect, housing 1402 is manufactured from a rigid, lightweight material such as plastic. Housing 1402 can be formed from upper half 1430 and lower half 1432 that are secured together (e.g. by glue, heat welding, snap-fit connection, etc.) after diversion pad 1404, sample pad 1406, and remainder 1408 of assay flow path, supported by backing 1420, have been placed in the lower half 1432 and covered by upper half 1430.

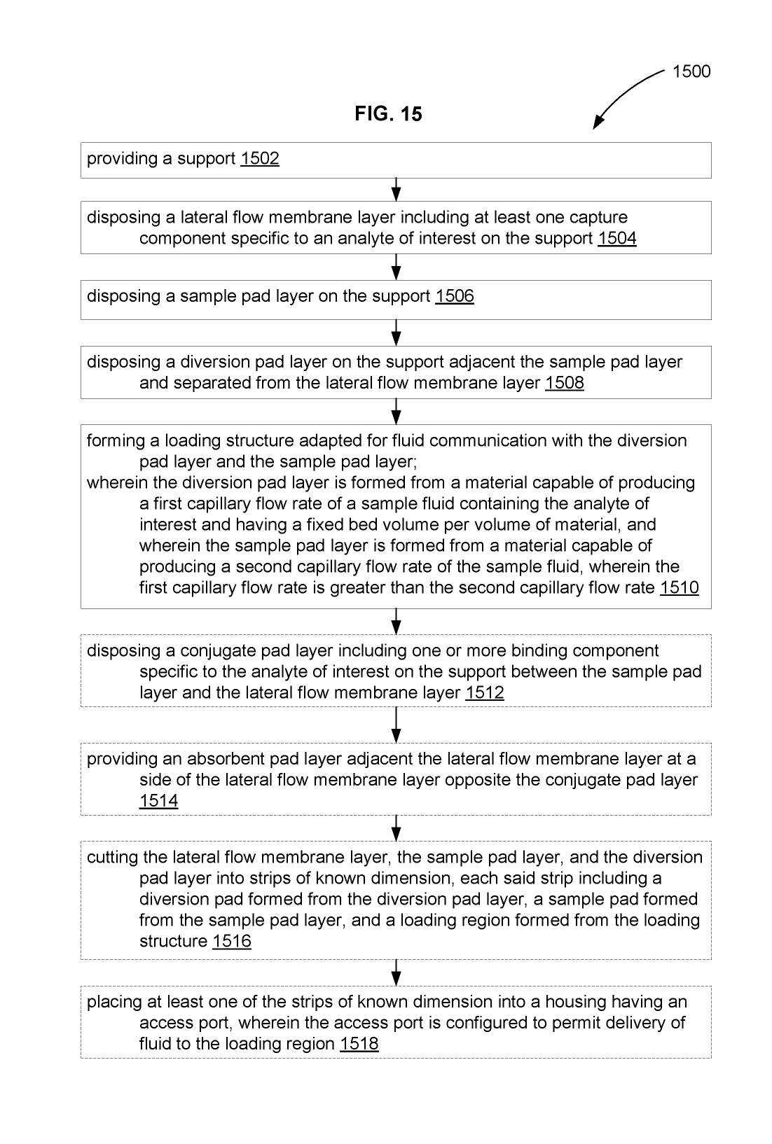

FIG. 15 illustrates a generalized method 1500 for manufacturing a lateral flow assay device of the type shown in FIGS. 5A and 5B and elsewhere herein. Method 1500 is performed generally as described in Brendan O'Farrell, “Chapter 1. Evolution in Lateral Flow-Based Immunoassay Systems”, pp. 16-17, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., pp. 28-29, both of which are incorporated herein by reference. Method 1500 includes providing a support, at 1502; disposing a lateral flow membrane layer including at least one capture component specific to an analyte of interest on the support, at 1504; disposing a sample pad layer on the support, at 1506; disposing a diversion pad layer on the support adjacent the sample pad layer and separated from the lateral flow membrane layer at 1508; and forming a loading structure adapted for fluid communication with the diversion pad layer and the sample pad layer, at 1510; wherein the diversion pad layer is formed from a material capable of producing a first capillary flow rate of a sample fluid containing the analyte of interest and having a fixed bed volume per volume of material, and wherein the sample pad layer is formed from a material capable of producing a second capillary flow rate of the sample fluid, wherein the first capillary flow rate is greater than the second capillary flow rate. In an aspect, providing the support, at 1502, includes providing a backing to which the lateral flow membrane layer, the sample pad layer, and the diversion pad layer are attached. In an aspect, the backing is an adhesive card. In other aspects, providing a support includes providing a rigid surface that supports the lateral flow assay device components during the manufacturing process but is not itself a component of the lateral flow assay device. Such a support can be used when the lateral flow assay device components are sufficiently rigid to be self-supporting.

In some aspects, method 1500 includes providing additional layers to form various lateral flow assay structures as discussed herein above. For example, in an aspect method 1500 includes disposing a conjugate pad layer including one or more binding component specific to the analyte of interest on the support between the sample pad layer and the lateral flow membrane layer, as indicated at 1512. In some aspects, method 1500 includes providing an absorbent pad layer adjacent the lateral flow membrane layer at a side of the lateral flow membrane layer opposite the conjugate pad layer, as indicated at 1514. Additional method steps include cutting the lateral flow membrane layer, the sample pad layer, and the diversion pad layer into strips of known dimension, each said strip including a diversion pad formed from the diversion pad layer, a sample pad formed from the sample pad layer, and a loading region formed from the loading structure, as indicated at 1516, and placing at least one of the strips of known dimension into a housing having an access port, wherein the access port is configured to permit delivery of fluid to the loading region, as indicated at 1518, for example as depicted in FIGS. 14A and 14B.

In an aspect, forming the loading structure, at 1510, includes disposing a loading pad layer between the sample pad layer and the diversion pad layer on the support, to form structures as depicted in FIGS. 12 and 13, for example. In an aspect, forming the loading structure, at 1510 includes abutting the sample pad layer against the diversion pad layer at a junction, as depicted in FIG. 5B, for example. In another aspect, forming the loading structure, at 1510, includes disposing a loading pad layer above the junction of the diversion pad layer and sample pad layer, overlapping both the diversion pad layer and the sample pad layer, to form structures as depicted in FIGS. 8 and 9, for example. In an aspect, method 1500 includes disposing a fluid impermeable barrier at the junction of the diversion pad layer and the sample pad layer, as depicted in FIG. 10. This can be done, for example, by printing, vapor deposition, or placement of a strip of sheet or film material at the junction. In an aspect, the material is placed into a gap between the diversion pad layer and the sample pad layer at the junction.

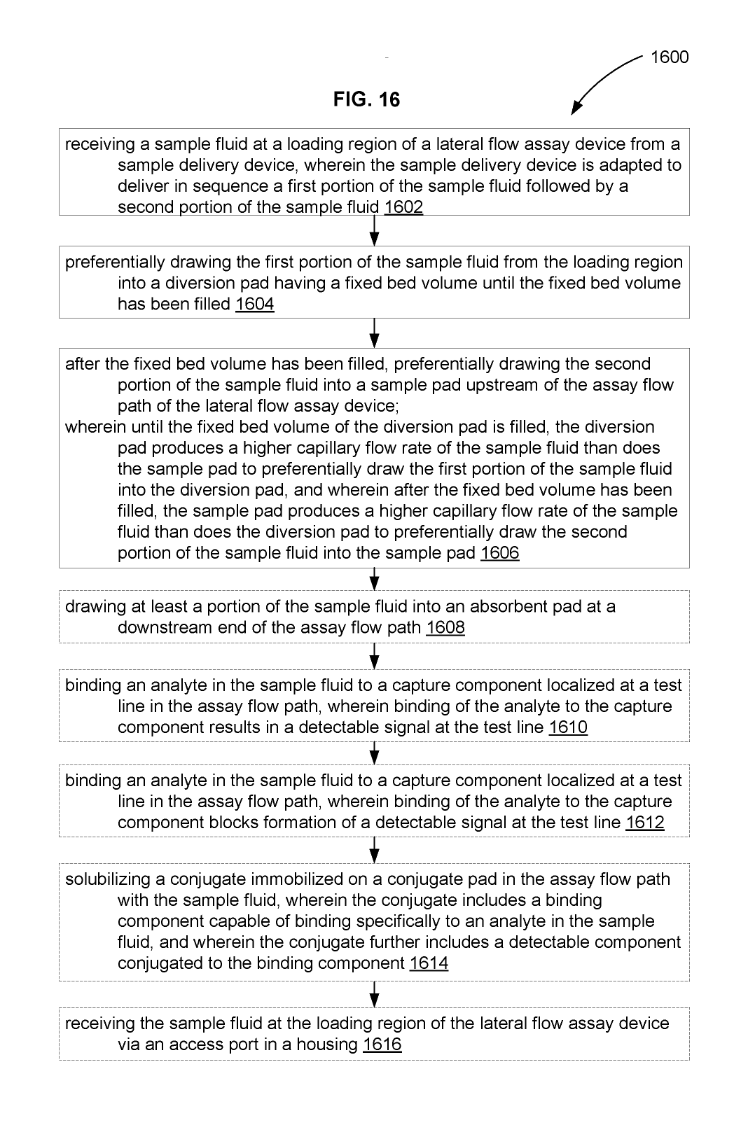

FIG. 16 is a flow diagram of a method 1600 for diverting a portion of a sample fluid away from an assay flow path of a lateral flow assay device, which can be performed with lateral flow assays as described herein. Method 1600 includes receiving a sample fluid at a loading region of a lateral flow assay device from a sample delivery device, wherein the sample delivery device is adapted to deliver in sequence a first portion of the sample fluid followed by a second portion of the sample fluid, at 1602; preferentially drawing the first portion of the sample fluid from the loading region into a diversion pad having a fixed bed volume until the fixed bed volume has been filled, at 1604; and after the fixed bed volume has been filled, preferentially drawing the second portion of the sample fluid into a sample pad upstream of the assay flow path of the lateral flow assay device; wherein until the fixed bed volume of the diversion pad is filled, the diversion pad produces a higher capillary flow rate of the sample fluid than does the sample pad to preferentially draw the first portion of the sample fluid into the diversion pad, and wherein after the fixed bed volume has been filled, the sample pad produces a higher capillary flow rate of the sample fluid than does the diversion pad to preferentially draw the second portion of the sample fluid into the sample pad, at 1606.

In an aspect, the first portion includes a portion of the sample fluid not desired for assay in the lateral flow assay, and the second portion includes a portion of the sample fluid desired for assay in the lateral flow assay. In some aspects, the volume of the portion of the sample fluid not desired for assay in the lateral flow assay is substantially equal to the fixed bed volume of the diversion pad, for example as depicted in FIG. 7A. In some aspects, the volume of the portion of the sample fluid not desired for assay in the lateral flow assay is less than the fixed bed volume of the diversion pad, for example as depicted in FIG. 7B. In some aspects, the volume of the portion of the sample fluid not desired for assay in the lateral flow assay is greater than the fixed bed volume of the diversion pad for example, such that it may enter the assay flow path, similar to the situation depicted in FIGS. 4A and 4B.

In some aspects of method 1600, receiving the sample fluid at the loading region of a lateral flow assay device, at 1602, includes receiving the sample fluid at a junction of the diversion pad and the sample pad (e.g., as depicted in FIGS. 6A-6C and 7A-7C). In other aspects of method 1600, receiving the sample fluid at the loading region of a lateral flow assay device, at 1602, includes receiving the sample fluid at a loading pad positioned above a junction of the diversion pad and the sample pad, wherein the loading pad overlaps both the diversion pad and the sample pad, as depicted in FIGS. 8 and 9.

In some aspects of method 1600, receiving the sample fluid at the loading region of a lateral flow assay device, at 1602, includes receiving the sample fluid at a loading pad located between the diversion pad and the sample pad (e.g., as depicted in FIGS. 12 and 13), and wherein preferentially drawing the first portion of the sample fluid from the loading region into a diversion pad includes drawing fluid from a first side of the loading pad into the diversion pad; and wherein preferentially drawing the second portion of the sample fluid into the sample pad includes drawing fluid from a second side of the loading pad. In some aspects, method 1600 includes drawing at least a portion of the sample fluid into an absorbent pad at a downstream end of the assay flow path, as indicated at 1608.

In some aspects, method 1600 includes binding an analyte in the sample fluid to a capture component localized at a test line in the assay flow path, wherein binding of the analyte to the capture component results in a detectable signal at the test line, as indicated at 1610. In other aspects, method 1600 includes binding an analyte in the sample fluid to a capture component localized at a test line in the assay flow path, wherein binding of the analyte to the capture component blocks formation of a detectable signal at the test line, as indicated at 1612.

In some aspects, method 1600 includes solubilizing a conjugate immobilized on a conjugate pad in the assay flow path with the sample fluid, wherein the conjugate includes a binding component capable of binding specifically to an analyte in the sample fluid, and wherein the conjugate further includes a detectable component conjugated to the binding component, as indicated at 1614. For example, in an aspect, method 1600 includes binding an analyte in the sample fluid to the binding component of the solubilized conjugate, and binding the analyte to capture component localized at a test line in the assay flow path, wherein binding of the analyte to the capture component results in a detectable signal from the detectable component at the test line. Method 1600 may additionally include binding the solubilized conjugate to a second capture component at a control line in the assay flow path to produce a detectable signal from the detectable component at the control line.

In an aspect, method 1600 includes receiving the sample fluid at the loading region of the lateral flow assay device via an access port in a housing, as indicated at 1616, for example as depicted in FIGS. 14A and 14B.

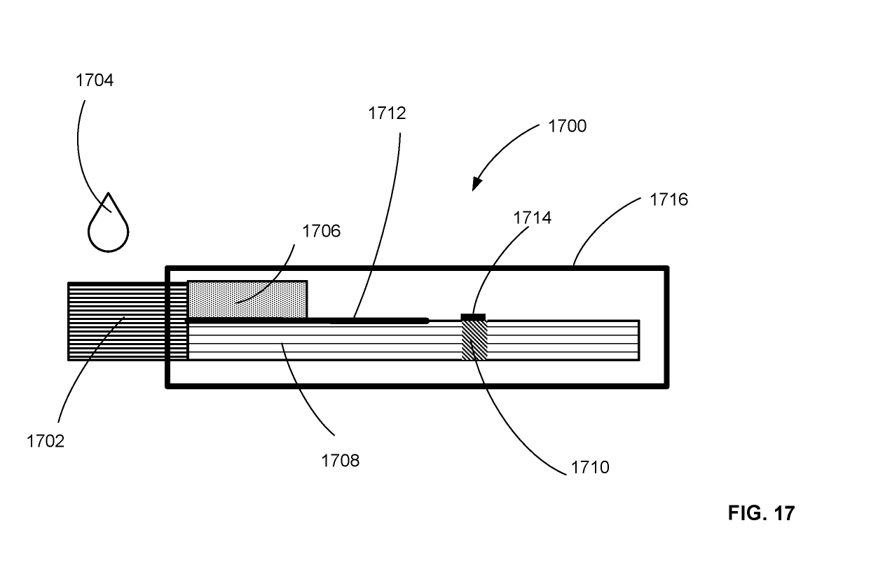

In various aspects, a diversion pad or related diversion structure is used in connection with other types of assay devices, not limited to lateral flow assay devices. FIG. 17 depicts a fluidic assay device 1700 that includes a loading region 1702 adapted to receive a sample fluid 1704; a diversion structure 1706 in fluid communication with the loading region 1702, the diversion structure having a fixed bed volume and capable of producing a first capillary flow rate of the sample fluid; a sample processing portion 1708 in fluid communication with the loading region 1702, the sample processing portion capable of producing a second capillary flow rate of the sample fluid, wherein the first capillary flow rate is greater than the second capillary flow rate; and a detection region 1710 within sample processing portion 1708, the detection region adapted to detect an analyte of interest within the sample fluid. In various aspects, sample processing portion 1708 includes various types of fluid handling structures. In some aspects, sample processing portion 1708 includes at least one microchannel. In some aspects, sample processing portion 1708 includes at least one capillary. For example, devices with capillary/microchannels include disposable glucometer test strips, blood lipid profilers, and other devices that accept glass capillary tubes containing collected blood samples. In some aspects, sample processing portion 1708 includes a porous material. In some aspects, sample processing portion 1708 includes at least one fluidic pathway. In an aspect, sample processing portion includes a lateral flow assay, and wherein the detection region includes a capture reagent. For example, in various aspects the assay includes an immunoassay, a quantitative assay, a sandwich assay, a competitive binding assay, or an inhibition assay.

In general, the design considerations for the relative capillary flow rates of diversion structure 1706 and sample processing portion 1708 are as discussed herein above. In addition, various aspects of device components and configurations not specifically discussed in connection with FIG. 17 are generally as discussed in connection with the various lateral flow assay devices described herein above.

In some aspects, loading region 1702 can receive the sample fluid from a fluid delivery device, such as a pipette, as described above, or from an automated sample delivery device. In an aspect, loading region 1702 includes a wick configured to receive the sample fluid from a urine stream. A wick can be formed from fibrous materials capable of rapidly drawing sample fluid into the loading region, for example as used for absorbent pad 214, as described herein above, such as cellulose, high-density cellulose, glass, polyester, nylon, cotton, mono-component fiber, or bi-component fiber (see, e.g., Brendan O'Farrell, “Chapter 1. Evolution in Lateral Flow-Based Immunoassay Systems”, in Lateral Flow Immunoassay, Raphael C. Wong and Harley Y. Tse, Editors, © 2009 ISBN: 978-1-58829-908-6 e-ISBN: 978-1-59745-240-3 DOI 10.1007/978-1-59745-240-3, and “Rapid Lateral Flow Test Strips: Considerations for Product Development,” Lit. No. TB500EN00EM Rev. C 12/13, © 2013, EMD Millipore Corporation, Billerica, Mass., both of which are incorporated herein by reference). In an aspect, loading region 1702 includes a wick configured to receive the sample fluid from a receptacle, such as a cup or the like.

In an aspect, fluidic assay device 1700 includes a fluid impermeable barrier 1712 between diversion structure 1706 and sample processing portion 1708. In an aspect, detection region 1710 includes at least one sensor 1714. Sensor 1714 may be, for example, a capillary based sensor. In an aspect, fluidic assay device 1700 includes a housing 1716, which is a cartridge similar to that depicted in FIGS. 14A and 14B. In other instances, housing 1716 is an instrument case or other housing designed to contain fluidic assay device 1700.

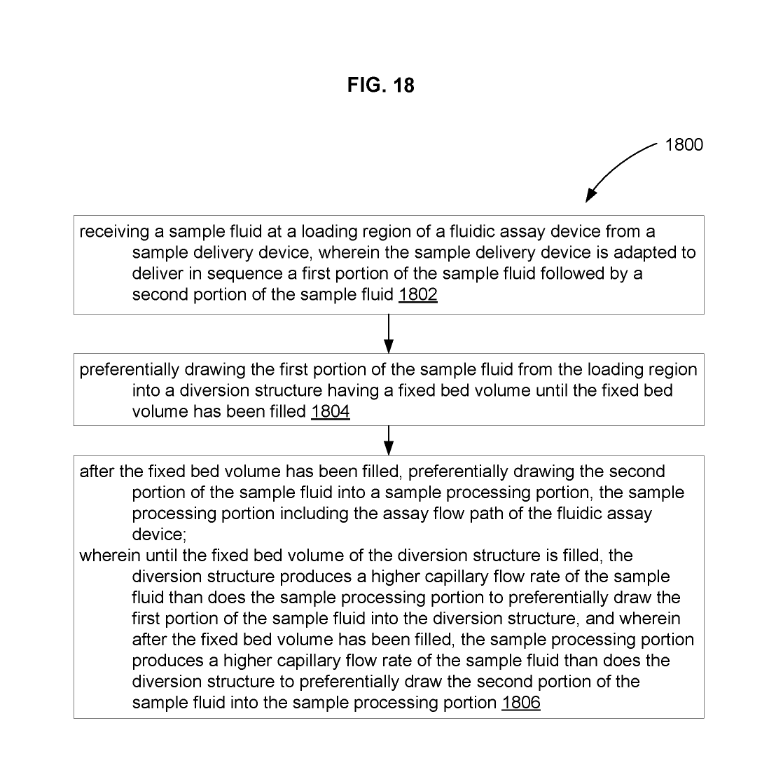

FIG. 18 depicts a method 1800 of diverting a portion of a sample fluid away from an assay flow path of a fluidic assay device. For example, method 1800 can be carried out in connection with a fluidic assay device 1700 as described in connection with FIG. 17. Method 1800 includes receiving a sample fluid at a loading region of a fluidic assay device from a sample delivery device, wherein the sample delivery device is adapted to deliver in sequence a first portion of the sample fluid followed by a second portion of the sample fluid, at 1802; preferentially drawing the first portion of the sample fluid from the loading region into a diversion structure having a fixed bed volume until the fixed bed volume has been filled, at 1804; and after the fixed bed volume has been filled, preferentially drawing the second portion of the sample fluid into a sample processing portion, the sample processing portion including the assay flow path of the fluidic assay device, wherein until the fixed bed volume of the diversion structure is filled, the diversion structure produces a higher capillary flow rate of the sample fluid than does the sample processing portion to preferentially draw the first portion of the sample fluid into the diversion structure, and wherein after the fixed bed volume has been filled, the sample processing portion produces a higher capillary flow rate of the sample fluid than does the diversion structure to preferentially draw the second portion of the sample fluid into the sample processing portion, at 1806.

### Example: TB-LAM Lateral Flow Assay System

In an aspect, a lateral flow assay device including a diversion pad is used for diagnosing tuberculosis (TB) by detecting of Mycobacterium tuberculosis cell wall antigen lipoarabinomannan (LAM) in urine. In an aspect, a urine sample is collected and concentrated by ultrafiltration in a small, benchtop dead end filtration device. The resulting concentrated urine sample is taken from the top of the filter and manually transferred to a lateral flow assay device, using a pipette. (As an alternative, concentrated sample could be transferred to the lateral flow assay device in automated fashion by being flushed from the concentrator and applied directly to the lateral flow assay device.) The first portion of the sample typically has not been concentrated to the same extent as the bulk of the sample. Therefore, it is desirable to discard the first portion of the sample. Modifying the design of a conventional lateral flow assay device to include a diversion pad for diverting the first portion of the sample away from the lateral flow assay device, allows the first portion of sample to be discarded without creating an additional step for the operator.

In an aspect, the concentrated urine sample is at least about 180 μl. In an aspect, between about 250 μl and about 300 μl of concentrated urine is collected. In an aspect, the first portion of the sample, having a non-representative (insufficient) concentration can be expected to between about 30 μl and about 80 μl of sample. Thus, between about the first 30 μl and about the first 80 μl of sample should be discarded. Accordingly, in an aspect, the diversion pad is selected to have a bed volume that is equal to or greater than the expected non-representative portion of the sample is collected. For example, in an aspect, the bed volume of the diversion pad is selected to be 90 μl. The bed volume of the diversion pad is the volume of fluid required to wet out the diversion pad, and is equal to the total volume of the diversion pad multiplied by the porosity of the pad.

Lateral flow assays for detecting LAM in urine typically are roughly 5 mm wide by 80 mm long, with thickness of the assay flow path varying along the length depending on the number of layers of material at a given point. The detection chemistry is generally as described in Lawn, S. D. (2012) “Point-of-care detection of lipoarabinomananan (LAM) in urine for diagnosis of HIV-associated tuberculosis: a state of the art review,” BMC Infections Diseases, 12:103, which is incorporated herein by reference. See also Lawn, S. D., Dheda, K., Kerkhoff, A. D., Peter, J. G., Dorman, Boehme, C. C., and Nicol, M. P., (2013), “Determine TB-LAM lateral flow urine antigen assay for HIV-associated tuberculosis: recommendations on the design and reporting of clinical studies,” BMC Infectious Diseases 13:407, which is also incorporated herein by reference. Typically, the thickest portion of the strip is the absorbent pad (e.g., absorbent pad 520 as depicted in FIGS. 5A and 5B), which in an aspect is about 2.5 mm thick. In an aspect, the TB-LAM strip is packaged within a cassette having outer dimension of 20 mm by 100 mm by 7 mm.

The design of the lateral flow assay device is modified to include the diversion pad upstream of the assay flow path. In an aspect, the dimensions and materials used in the construction of the assay flow path are unchanged. The diversion pad width is selected to match the width of the sample pad and other components of the lateral flow assay device. A diversion pad material is selected that has a capillary flow time that is less than the capillary flow time for the sample pad (this means that the capillary flow rate for the diversion pad will be higher than the capillary flow rate for the sample pad). The length and thickness of the diversion pad is determined that will provide a bed volume equal to or greater than the expected non-representative first portion of the sample. In an aspect, pad materials are available in several thicknesses, and a thicker pad material may be selected to reduce the length of the pad (and thus the length of the assay device as a whole). In an aspect, the dimension of the backing is increased to provide support for the diversion pad. Alternatively, the dimensions of the assay flow path may be reduced to accommodate the added length of the diversion pad while maintaining the same overall length of the assay device. Design of the lateral flow assay device may be performed, for example, according to the principles outlined in Hsieh, H. V, Dantzler, J. L. and Weigl, B. H, (2017) “Analytical Tools to Improve Optimization Procedures for Lateral Flow Assays,” Diagnostics, 7, 29; doi:10.3390/diagnostics7020029, which is incorporated herein by reference.

Aspects of the subject matter described herein are set out in the following numbered clauses:

### Clauses

1. A lateral flow assay device comprising:
a diversion pad having a fixed bed volume and capable of producing a first capillary flow rate of a sample fluid;
a sample pad capable of producing a second capillary flow rate of the sample fluid;
a loading region adapted to receive the sample fluid, wherein the loading region is configured for fluid communication with the diversion pad and the sample pad; and
a lateral flow membrane downstream of the sample pad and including one or more capture component adapted to capture an analyte of interest in the sample fluid;
wherein the first capillary flow rate is greater than the second capillary flow rate.
2. The lateral flow assay device of clause 1, wherein at least one of the capture component, a dimension of the lateral flow membrane, and a flow rate of the lateral flow membrane are optimized for performing a quantitative assay for the analyte of interest.
3. The lateral flow assay device of clause 1, wherein the diversion pad and the sample pad abut each other at a junction.
4. The lateral flow assay device of clause 3, wherein the loading region includes the junction between the diversion pad and the sample pad.
5. The lateral flow assay device of clause 3, wherein the loading region includes a loading pad positioned above the junction of the diversion pad and the sample pad, wherein the loading pad overlaps and is in fluid communication with both the diversion pad and the sample pad.
6. The lateral flow assay device of clause 5, wherein the loading pad is formed from cellulose, high-density cellulose, glass, polyester, nylon, cotton, mono-component fiber, or bi-component fiber.
7. The lateral flow assay device of clause 5, wherein the loading pad is capable of producing a third capillary flow rate of the sample fluid, wherein the third capillary flow rate is higher than the first capillary flow rate and the second capillary flow rate.
8. The lateral flow assay device of clause 5, wherein the loading pad is positioned asymmetrically over the diversion pad and the sample pad, with greater overlap of the diversion pad than the sample pad.
9. The lateral flow assay device of clause 5, including a fluid impermeable barrier between the diversion pad and the sample pad.
10. The lateral flow assay device of clause 5, including a flow limiting structure between the diversion pad and the sample pad.
11. The lateral flow assay device of clause 10, wherein the flow limiting structure includes a pinch point.
12. The lateral flow assay device of clause 10, wherein the flow limiting structure includes a dam formed from a dissolvable material, wherein the dissolvable material is dissolvable by the sample fluid.
13. The lateral flow assay device of clause 10, wherein the flow limiting structure includes a hydrophilic region.
14. The lateral flow assay device of clause 10, wherein the flow limiting structure includes a hydrophobic region.
15. The lateral flow assay device of clause 1, wherein the loading region includes a loading pad located between the diversion pad and the sample pad and abutting the diversion pad on a first side of the loading pad and abutting the sample pad on a second side of the loading pad.
16. The lateral flow assay device of clause 15, wherein the loading pad is wider at a top surface and narrower at a bottom region, such that at least one of the surface abutting the sample pad and the surface abutting the diversion pad angle is angled with respect to the top surface of the loading pad and wider than the thickness of the loading pad.
17. The lateral flow assay device of clause 1, including
a housing configured to contain the diversion pad, the sample pad, and the lateral flow membrane; and an access port in the housing configured to permit delivery of fluid to the loading region.
18. The lateral flow assay device of clause 1, including a backing on which the diversion pad, sample pad, and lateral flow membrane are supported.
19. The lateral flow assay device of clause 18, wherein the backing includes at least one of a non-porous plastic, polystyrene, vinyl, or polyester.
20. The lateral flow assay device of clause 1, wherein the diversion pad includes at least one of cellulose, glass fiber, cotton, rayon, a woven mesh, and a synthetic non-woven material.
21. The lateral flow assay device of clause 1, wherein the diversion pad includes at least one superabsorbent material.
22. The lateral flow assay device of clause 1, wherein the sample pad includes at least one of cellulose, glass fiber, cotton, rayon, a woven mesh, and a synthetic non-woven material.
23. The lateral flow assay device of clause 1, wherein the sample pad includes at least one of a protein, a detergent, a viscosity enhance, a buffer, or a salt for modifying at least one property of the sample fluid.
24. The lateral flow assay device of clause 1, wherein the lateral flow membrane includes at least one of nitrocellulose, polyvinylidene fluoride, charge-modified nylon, polyether sulfone, nitrocellulose acetate, glass fiber, cellulose, paper, silica, a porous synthetic polymer, polyester, nylon, cotton, a sintered material, a woven material, or a non-woven material.
25. The lateral flow assay device of clause 1, wherein the bed volume of the diversion pad is sufficient to contain an expected first sample volume.
26. The lateral flow assay device of clause 1, including a conjugate pad containing an immobilized conjugate, wherein the conjugate includes a binding component adapted to bind to the analyte of interest in the fluid, and a detectable component conjugated to the binding component.
27. The lateral flow assay device of clause 26, wherein the binding component includes an antibody to the analyte.
28. The lateral flow assay device of clause 26, wherein the detectable component includes at least one of a latex bead, a colloidal metal, a colloidal gold particle, colloidal carbon, a fluorescent label, a luminescent label, a quantum dot, an upconverting phosphore, a bioluminescent marker, an enzyme, a magnetic particle, a paramagnetic particles, a dye, or an electroactive compound.
29. The lateral flow assay device of clause 26, wherein the conjugate pad includes glass fiber.
30. The lateral flow assay device of clause 26, wherein at least one of the binding component and the detectable component is optimized for performing a quantitative assay for the analyte of interest.
31. The lateral flow assay device of clause 26, wherein the binding component is adapted to bind a marker related to inflammation.
32. The lateral flow assay device of clause 26, wherein the binding component is adapted to bind a marker related to tissue damage.
33. The lateral flow assay device of clause 26, wherein the binding component is adapted to bind a marker related to blood vessel damage.
34. The lateral flow assay device of clause 26, wherein the binding component is adapted to bind C-Reactive Protein.
35. The lateral flow assay device of clause 26, wherein the binding component is adapted to bind soluble triggering receptor expressed on myeloid cells.
36. The lateral flow assay device of clause 26, wherein the binding component is adapted to bind a marker affected by at least one of oxygen concentration, carbon dioxide concentration, and pH.
37. The lateral flow assay device of clause 30, wherein the binding component is adapted to bind a marker related to sepsis.
38. The lateral flow assay device of clause 30, wherein the binding component is adapted to bind a marker related to at least one of enteric bacteria, mycobacteria, or coliform bacteria.
39. The lateral flow assay device of clause 30, wherein the binding component is adapted to bind tuberculosis Mycobacterium tuberculosis cell wall antigen lipoarabinomannan.
40. The lateral flow assay device of clause 1, including an absorbent pad located downstream of the lateral flow membrane and adapted to receive fluid that has passed through the lateral flow membrane.
41. The lateral flow assay device of clause 40, wherein the absorbent pad includes at least one of cellulose, high-density cellulose, glass, polyester, nylon, cotton, mono-component fiber, or bi-component fiber.
42. The lateral flow assay device of clause 1, wherein the capture component is located at a test line on the lateral flow membrane.
43. A method of manufacturing a lateral flow assay device, comprising:
providing a support;
disposing a lateral flow membrane layer including at least one capture component specific to an analyte of interest on a support;
disposing a sample pad layer on the support;
disposing a diversion pad layer on the support adjacent the sample pad layer and separated from the lateral flow membrane layer; and
forming a loading structure adapted for fluid communication with the diversion pad layer and the sample pad layer;
wherein the diversion pad layer is formed from a material capable of producing a first capillary flow rate of a sample fluid containing the analyte of interest and having a fixed bed volume per volume of material and, and wherein the sample pad layer is formed from a material capable of producing a second capillary flow rate of the sample fluid, wherein the first capillary flow rate is greater than the second capillary flow rate.
44. The method of clause 43, including disposing a conjugate pad layer including one or more binding component specific to the analyte of interest on the support between the sample pad layer and the lateral flow membrane layer.
45. The method of clause 44, wherein the one or more binding component is component is optimized for performing a quantitative assay for the analyte of interest.
46. The method of clause 44, wherein at least one of the binding component and the capture component is adapted to bind a marker related to inflammation.
47. The method of clause 44, wherein at least one of the binding component and the capture component is adapted to bind a marker related to tissue damage.
48. The method of clause 44, wherein at least one of the binding component and the capture component is adapted to bind a marker related to blood vessel damage.
49. The method of clause 44, wherein at least one of the binding component and the capture component is adapted to bind C-Reactive Protein.
50. The method of clause 44, wherein at least one of the binding component and the capture component is adapted to bind soluble triggering receptor expressed on myeloid cells.
51. The method of clause 44, wherein at least one of the binding component and the capture component is adapted to bind a marker affected by at least one of oxygen concentration, carbon dioxide concentration, and pH.
52. The method of clause 44, wherein at least one of the binding component and the capture component is adapted to bind a marker related to sepsis.
53. The method of clause 44, wherein at least one of the binding component and the capture component is adapted to bind a marker related to at least one of enteric bacteria, mycobacteria, or coliform bacteria.
54. The method of clause 44, wherein at least one of the binding component and the capture component is adapted to bind tuberculosis Mycobacterium tuberculosis cell wall antigen lipoarabinomannan.
55. The method of clause 43, wherein providing the support includes providing a backing to which the lateral flow membrane layer, the sample pad layer, and the diversion pad layer are attached.
56. The method of clause 55, wherein providing the backing includes providing an adhesive card.
57. The method of clause 43, including cutting the lateral flow membrane layer, the sample pad layer, and the diversion pad layer into strips of known dimension, each said strip including a diversion pad formed from the diversion pad layer, a sample pad formed from the sample pad layer, and a loading region formed from the loading structure.
58. The method of clause 57, including placing at least one of the strips of known dimension into a housing having an access port, wherein the access port is configured to permit delivery of fluid to the loading region.
59. The method of clause 43, including providing an absorbent pad layer adjacent the lateral flow membrane layer at a side of the lateral flow membrane layer opposite the conjugate pad layer.
60. The method of clause 43, wherein forming the loading structure includes disposing a loading pad layer between the sample pad layer and the diversion pad layer on the support.
61. The method of clause 43, wherein forming the loading structure includes abutting the sample pad layer against the diversion pad layer at a junction.
62. The method of clause 61, wherein forming the loading structure includes disposing a loading pad layer above the junction of the diversion pad layer and sample pad layer, overlapping both the diversion pad layer and the sample pad layer.
63. The method of clause 62, including disposing a fluid impermeable barrier at the junction of the diversion pad layer and the sample pad layer.
64. The method of clause 62, including disposing a flow limiting structure between the diversion pad and the sample pad.
65. The method of clause 64, wherein the flow limiting structure includes a pinch point.
66. The method of clause 64, wherein the flow limiting structure includes a dam formed from a dissolvable material, wherein the dissolvable material is dissolvable by the sample fluid.
67. The method of clause 64, wherein the flow limiting structure includes a hydrophilic region.
68. The method of clause 64, wherein the flow limiting structure includes a hydrophobic region.
69. A method of diverting a portion of a sample fluid away from an assay flow path of a lateral flow assay device, comprising:
receiving a sample fluid at a loading region of a lateral flow assay device from a sample delivery device, wherein the sample delivery device is adapted to deliver in sequence a first portion of the sample fluid followed by a second portion of the sample fluid;
preferentially drawing the first portion of the sample fluid from the loading region into a diversion pad having a fixed bed volume until the fixed bed volume has been filled; and
after the fixed bed volume has been filled, preferentially drawing the second portion of the sample fluid into a sample pad upstream of the assay flow path of the lateral flow assay device;
wherein until the fixed bed volume of the diversion pad is filled, the diversion pad produces a higher capillary flow rate of the sample fluid than does the sample pad to preferentially draw the first portion of the sample fluid into the diversion pad, and wherein after the fixed bed volume has been filled, the sample pad produces a higher capillary flow rate of the sample fluid than does the diversion pad to preferentially draw the second portion of the sample fluid into the sample pad.
70. The method of clause 69, wherein the first portion includes a portion of the sample fluid not desired for assay in the lateral flow assay, and wherein the second portion includes a portion of the sample fluid desired for assay in the lateral flow assay.
71. The method of clause 70, wherein the volume of the portion of the sample fluid not desired for assay in the lateral flow assay is substantially equal to the fixed bed volume of the diversion pad.
72. The method of clause 70, wherein the volume of the portion of the sample fluid not desired for assay in the lateral flow assay is less than the fixed bed volume of the diversion pad.
73. The method of clause 70, wherein the volume of the portion of the sample fluid not desired for assay in the lateral flow assay is greater than the fixed bed volume of the diversion pad.
74. The method of clause 69, wherein receiving the sample fluid at the loading region of a lateral flow assay device includes receiving the sample fluid at a junction of the diversion pad and the sample pad.
75. The method of clause 69, wherein receiving the sample fluid at the loading region of a lateral flow assay device includes receiving the sample fluid at a loading pad positioned above a junction of the diversion pad and the sample pad, wherein the loading pad overlaps both the diversion pad and the sample pad.
76. The method of clause 69, wherein receiving the sample fluid at the loading region of a lateral flow assay device includes receiving the sample fluid at a loading pad located between the diversion pad and the sample pad; wherein preferentially drawing the first portion of the sample fluid from the loading region into the diversion pad includes drawing fluid from a first side of a loading pad into the diversion pad; and wherein preferentially drawing the second portion of the sample fluid into the sample pad includes drawing fluid from a second side of the loading pad.
77. The method of clause 69, including drawing at least a portion of the sample fluid into an absorbent pad at a downstream end of the assay flow path.
78. The method of clause 69, including binding an analyte in the sample fluid to a capture component localized at a test line in the assay flow path, wherein binding of the analyte to the capture component results in a detectable signal at the test line.
79. The method of clause 78, wherein binding of the analyte to the capture component results in a quantifiable detectable signal at the test line.
80. The method of clause 78, wherein binding the analyte in the sample fluid includes binding a marker related to inflammation.
81. The method of clause 78, wherein binding the analyte in the sample fluid includes binding a marker related to tissue damage.
82. The method of clause 78, wherein binding the analyte in the sample fluid includes binding a marker related to blood vessel damage.
83. The method of clause 78, wherein binding the analyte in the sample fluid includes binding C-Reactive Protein.
84. The method of clause 78, wherein binding the analyte in the sample fluid includes binding soluble triggering receptor expressed on myeloid cells.
85. The method of clause 78, wherein binding the analyte in the sample fluid includes binding a marker affected by at least one of oxygen concentration, carbon dioxide concentration, and pH.
86. The method of clause 78, wherein binding the analyte in the sample fluid includes binding a marker related to sepsis.
87. The method of clause 78, wherein binding the analyte in the sample fluid includes binding a marker related to at least one of enteric bacteria, mycobacteria, or coliform bacteria.
88. The method of clause 78, wherein binding the analyte in the sample fluid includes binding tuberculosis Mycobacterium tuberculosis cell wall antigen lipoarabinomannan.
89. The method of clause 69, including binding an analyte in the sample fluid to a capture component localized at a test line in the assay flow path, wherein binding of the analyte to the capture component blocks formation of a detectable signal at the test line.
90. The method of clause 69, including solubilizing a conjugate immobilized on a conjugate pad in the assay flow path with the sample fluid, wherein the conjugate includes a binding component capable of binding specifically to an analyte in the sample fluid, and wherein the conjugate further includes a detectable component conjugated to the binding component.
91. The method of clause 90, binding an analyte in the sample fluid to the binding component of the solubilized conjugate, and binding the analyte to capture component localized at a test line in the assay flow path, wherein binding of the analyte to the capture component results in a detectable signal from the detectable component at the test line.
92. The method of clause 91, including the binding the solubilized conjugate to a second capture component at a control line in the assay flow path to produce a detectable signal from the detectable component at the control line.
93. The method of clause 69, including receiving the sample fluid at the loading region of the lateral flow assay device via an access port in a housing.
94. A fluidic assay device comprising:
a loading region adapted to receive a sample fluid;
a diversion structure in fluid communication with the loading region, the diversion structure having a fixed bed volume and capable of producing a first capillary flow rate of the sample fluid;
a sample processing portion in fluid communication with the loading region, the sample processing portion capable of producing a second capillary flow rate of the sample fluid, wherein the first capillary flow rate is greater than the second capillary flow rate; and
a detection region within the sample processing portion, the detection region adapted to detect an analyte of interest within the sample fluid.
95. The fluidic assay device of clause 94, wherein the loading region is configured to receive the sample fluid from a fluid delivery device.
96. The fluidic assay device of clause 94, wherein the loading region includes a wick configured to receive the sample fluid from a urine stream.
97. The fluidic assay device of clause 94, wherein the loading region includes a wick configured to receive the sample fluid from a receptacle.
98. The fluidic assay device of clause 94, wherein the diversion structure and the sample processing portion abut each other at a junction, and wherein the loading region includes the junction between the diversion structure and the sample processing portion.
99. The fluidic assay device of clause 94, wherein the diversion structure and the sample processing portion abut each other at a junction, and wherein the loading region includes a loading pad positioned above the junction of the diversion structure and the sample processing portion, wherein the loading pad overlaps and is in fluid communication with both the diversion structure and the sample processing portion.
100. The fluidic assay device of clause 99, wherein the loading pad is formed from cellulose, high-density cellulose, glass, polyester, nylon, cotton, mono-component fiber, or bi-component fiber.
101. The fluidic assay device of clause 99, wherein the loading pad is capable of producing a third capillary flow rate of the sample fluid, wherein the third capillary flow rate is higher than the first capillary flow rate and the second capillary flow rate.
102. The fluidic assay device of clause 99, including a fluid impermeable barrier between the diversion structure and the sample processing portion.
103. The fluidic assay device of clause 99, including a flow limiting structure between the diversion structure and the sample processing portion.
104. The fluidic assay device of clause 103, wherein the flow limiting structure includes a pinch point.
105. The fluidic assay device of clause 103, wherein the flow limiting structure includes a dam formed from a dissolvable material, wherein the dissolvable material is dissolvable by the sample fluid.
106. The fluidic assay device of clause 103, wherein the flow limiting structure includes a hydrophilic region.
107. The fluidic assay device of clause 103, wherein the flow limiting structure includes a hydrophobic region. 108. The fluidic assay device of clause 94, wherein the loading region includes a loading pad located between the diversion structure and the sample processing portion and abutting the diversion structure on a first side of the loading pad and abutting the sample processing portion on a second side of the loading pad.
109. The fluidic assay device of clause 94, wherein the sample processing portion includes a lateral flow assay, and wherein the detection region includes a capture reagent.
110. The fluidic assay device of clause 109, wherein the lateral flow assay includes at least one of an immunoassay, a quantitative assay, a sandwich assay, a competitive, and an inhibition assay binding assay. 111. The fluidic assay device of clause 94, wherein the detection region includes at least one sensor.
112. The fluidic assay device of clause 111, wherein the at least one sensor includes at least one capillary based sensor.
113. The fluidic assay device of clause 94, wherein the sample processing portion includes at least one microchannel.
114. The fluidic assay device of clause 94, wherein the sample processing portion includes a porous material.
115. The fluidic assay device of clause 94, wherein the sample processing portion includes at least one fluidic pathway.
116. A method of diverting a portion of a sample fluid away from an assay flow path of a fluidic assay device, comprising:
receiving a sample fluid at a loading region of a fluidic assay device from a sample delivery device, wherein the sample delivery device is adapted to deliver in sequence a first portion of the sample fluid followed by a second portion of the sample fluid;
preferentially drawing the first portion of the sample fluid from the loading region into a diversion structure having a fixed bed volume until the fixed bed volume has been filled; and
after the fixed bed volume has been filled, preferentially drawing the second portion of the sample fluid into a sample processing portion, the sample processing portion including the assay flow path of the fluidic assay device;
wherein until the fixed bed volume of the diversion structure is filled, the diversion structure produces a higher capillary flow rate of the sample fluid than does the sample processing portion to preferentially draw the first portion of the sample fluid into the diversion structure, and wherein after the fixed bed volume has been filled, the sample processing portion produces a higher capillary flow rate of the sample fluid than does the diversion structure to preferentially draw the second portion of the sample fluid into the sample processing portion.
117. The method of clause 116, wherein the first portion includes a portion of the sample fluid not desired for assay in the lateral flow assay, and wherein the second portion includes a portion of the sample fluid desired for assay in the lateral flow assay.
118. The method of clause 116, wherein receiving the sample fluid at the loading region of a fluidic assay device includes receiving the sample fluid at a junction of the diversion structure and the sample processing portion.
119. The method of clause 116, wherein receiving the sample fluid at the loading region of a fluidic assay device includes receiving the sample fluid at a loading pad positioned above a junction of the diversion structure and the sample processing portion, wherein the loading pad overlaps both the diversion structure and the sample processing portion.
120. The method of clause 116, wherein receiving the sample fluid at the loading region of a fluidic assay device includes receiving the sample fluid at a loading pad located between the diversion structure and the sample processing portion; wherein preferentially drawing the first portion of the sample fluid from the loading region into the diversion structure includes drawing fluid from a first side of a loading pad into the diversion structure; and wherein preferentially drawing the second portion of the sample fluid into the sample processing portion includes drawing fluid from a second side of the loading pad.
121. The method of clause 116, including drawing at least a portion of the sample fluid into an absorbent pad at a downstream end of the assay flow path.
122. The method of clause 116, including binding an analyte in the sample fluid to a capture component localized at a test line in the assay flow path, wherein binding of the analyte to the capture component results in a detectable signal at the test line.
123. The method of clause 122, wherein binding of the analyte to the capture component results in a quantifiable detectable signal at the test line.
124. The method of clause 123, wherein binding the analyte in the sample fluid includes binding at least one of a marker related to inflammation, a marker related to tissue damage, a marker related to blood vessel damage, C-Reactive Protein, soluble triggering receptor expressed on myeloid cells, a marker affected by oxygen concentration, a marker affected by carbon dioxide concentration, a marker affected by pH, a marker related to sepsis, a marker related to enteric bacteria, a marker related to mycobacteria, a marker related to coliform bacteria, or tuberculosis Mycobacterium tuberculosis cell wall antigen lipoarabinomannan.
125. The method of clause 122, including binding an analyte in the sample fluid to a capture component localized at a test line in the assay flow path, wherein binding of the analyte to the capture component blocks formation of a detectable signal at the test line.

The herein described subject matter sometimes illustrates different components contained within, or connected with, different other components. It is to be understood that such depicted architectures are merely exemplary, and that in fact many other architectures may be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components, and/or wirelessly interactable, and/or wirelessly interacting components, and/or logically interacting, and/or logically interactable components.

In some instances, one or more components may be referred to herein as “configured to,” “configured by,” “configurable to,” “operable/operative to,” “adapted/adaptable,” “able to,” “conformable/conformed to,” etc. Those skilled in the art will recognize that such terms (e.g. “configured to”) generally encompass active-state components and/or inactive-state components and/or standby-state components, unless context requires otherwise.

The herein described components (e.g., operations), devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components (e.g., operations), devices, and objects should not be taken limiting.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A lateral flow assay device comprising:
a diversion pad having a fixed bed volume and capable of producing a first capillary flow rate of a sample fluid;
a sample pad capable of producing a second capillary flow rate of the sample fluid;
a loading region adapted to receive the sample fluid, wherein the loading region is configured for fluid communication with the diversion pad and the sample pad; and
a lateral flow membrane downstream of the sample pad and including one or more capture component adapted to capture an analyte of interest in the sample fluid;
wherein the first capillary flow rate is greater than the second capillary flow rate.

2. The lateral flow assay device of claim 1, wherein at least one of the capture component, a dimension of the lateral flow membrane, and a flow rate of the lateral flow membrane are optimized for performing a quantitative assay for the analyte of interest.

3. The lateral flow assay device of claim 1, wherein the diversion pad and the sample pad abut each other at a junction.

4. The lateral flow assay device of claim 3, wherein the loading region includes the junction between the diversion pad and the sample pad.

5. The lateral flow assay device of claim 3, wherein the loading region includes a loading pad positioned above the junction of the diversion pad and the sample pad, wherein the loading pad overlaps and is in fluid communication with both the diversion pad and the sample pad.

6. (canceled)

7. The lateral flow assay device of claim 5, wherein the loading pad is capable of producing a third capillary flow rate of the sample fluid, wherein the third capillary flow rate is higher than the first capillary flow rate and the second capillary flow rate.

8. (canceled)

9. The lateral flow assay device of claim 5, including a fluid impermeable barrier between the diversion pad and the sample pad.

10. The lateral flow assay device of claim 5, including a flow limiting structure between the diversion pad and the sample pad.

11.-14. (canceled)

15. The lateral flow assay device of claim 1, wherein the loading region includes a loading pad located between the diversion pad and the sample pad and abutting the diversion pad on a first side of the loading pad and abutting the sample pad on a second side of the loading pad.

16. (canceled)

17. The lateral flow assay device of claim 1, including
a housing configured to contain the diversion pad, the sample pad, and the lateral flow membrane; and
an access port in the housing configured to permit delivery of fluid to the loading region.

18.-24. (canceled)

25. The lateral flow assay device of claim 1, wherein the bed volume of the diversion pad is sufficient to contain an expected first sample volume.

26. The lateral flow assay device of claim 1, including a conjugate pad containing an immobilized conjugate, wherein the conjugate includes a binding component adapted to bind to the analyte of interest in the fluid, and a detectable component conjugated to the binding component.

27.-29. (canceled)

30. The lateral flow assay device of claim 26, wherein at least one of the binding component and the detectable component is optimized for performing a quantitative assay for the analyte of interest.

31.-42. (canceled)

43. A method of manufacturing a lateral flow assay device, comprising:
providing a support;
disposing a lateral flow membrane layer including at least one capture component specific to an analyte of interest on a support;
disposing a sample pad layer on the support;
disposing a diversion pad layer on the support adjacent the sample pad layer and separated from the lateral flow membrane layer; and
forming a loading structure adapted for fluid communication with the diversion pad layer and the sample pad layer;
wherein the diversion pad layer is formed from a material capable of producing a first capillary flow rate of a sample fluid containing the analyte of interest and having a fixed bed volume per volume of material and, and wherein the sample pad layer is formed from a material capable of producing a second capillary flow rate of the sample fluid, wherein the first capillary flow rate is greater than the second capillary flow rate.

44. The method of claim 43, including disposing a conjugate pad layer including one or more binding component specific to the analyte of interest on the support between the sample pad layer and the lateral flow membrane layer.

45. The method of claim 44, wherein the one or more binding component is component is optimized for performing a quantitative assay for the analyte of interest.

46.-54. (canceled)

55. The method of claim 43, wherein providing the support includes providing a backing to which the lateral flow membrane layer, the sample pad layer, and the diversion pad layer are attached.

56. (canceled)

57. The method of claim 43, including cutting the lateral flow membrane layer, the sample pad layer, and the diversion pad layer into strips of known dimension, each said strip including a diversion pad formed from the diversion pad layer, a sample pad formed from the sample pad layer, and a loading region formed from the loading structure.

58. The method of claim 57, including placing at least one of the strips of known dimension into a housing having an access port, wherein the access port is configured to permit delivery of fluid to the loading region.

59.-68. (canceled)

69. A method of diverting a portion of a sample fluid away from an assay flow path of a lateral flow assay device, comprising:
receiving a sample fluid at a loading region of a lateral flow assay device from a sample delivery device, wherein the sample delivery device is adapted to deliver in sequence a first portion of the sample fluid followed by a second portion of the sample fluid;
preferentially drawing the first portion of the sample fluid from the loading region into a diversion pad having a fixed bed volume until the fixed bed volume has been filled; and
after the fixed bed volume has been filled, preferentially drawing the second portion of the sample fluid into a sample pad upstream of the assay flow path of the lateral flow assay device;
wherein until the fixed bed volume of the diversion pad is filled, the diversion pad produces a higher capillary flow rate of the sample fluid than does the sample pad to preferentially draw the first portion of the sample fluid into the diversion pad, and wherein after the fixed bed volume has been filled, the sample pad produces a higher capillary flow rate of the sample fluid than does the diversion pad to preferentially draw the second portion of the sample fluid into the sample pad.

70.-93. (canceled)

94. A fluidic assay device comprising:
a loading region adapted to receive a sample fluid;
a diversion structure in fluid communication with the loading region, the diversion structure having a fixed bed volume and capable of producing a first capillary flow rate of the sample fluid;
a sample processing portion in fluid communication with the loading region, the sample processing portion capable of producing a second capillary flow rate of the sample fluid, wherein the first capillary flow rate is greater than the second capillary flow rate; and
a detection region within the sample processing portion, the detection region adapted to detect an analyte of interest within the sample fluid.

95.-115. (canceled)

116. A method of diverting a portion of a sample fluid away from an assay flow path of a fluidic assay device, comprising:
receiving a sample fluid at a loading region of a fluidic assay device from a sample delivery device, wherein the sample delivery device is adapted to deliver in sequence a first portion of the sample fluid followed by a second portion of the sample fluid;
preferentially drawing the first portion of the sample fluid from the loading region into a diversion structure having a fixed bed volume until the fixed bed volume has been filled; and
after the fixed bed volume has been filled, preferentially drawing the second portion of the sample fluid into a sample processing portion, the sample processing portion including the assay flow path of the fluidic assay device;
wherein until the fixed bed volume of the diversion structure is filled, the diversion structure produces a higher capillary flow rate of the sample fluid than does the sample processing portion to preferentially draw the first portion of the sample fluid into the diversion structure, and wherein after the fixed bed volume has been filled, the sample processing portion produces a higher capillary flow rate of the sample fluid than does the diversion structure to preferentially draw the second portion of the sample fluid into the sample processing portion.

117.-125. (canceled)

126. The lateral flow assay device of claim 3, wherein the loading region includes at least one of
the junction between the diversion pad and the sample pad; and
a loading pad positioned above the junction of the diversion pad and the sample pad, wherein the loading pad overlaps and is in fluid communication with both the diversion pad and the sample pad.

127. The lateral flow assay device of claim 126, wherein the loading pad is capable of producing a third capillary flow rate of the sample fluid, wherein the third capillary flow rate is higher than the first capillary flow rate and the second capillary flow rate.

128. The lateral flow assay device of claim 126, including at least one of a fluid impermeable barrier between the diversion pad and the sample pad; and a flow limiting structure between the diversion pad and the sample pad.

129. The lateral flow assay device of claim 128, wherein the flow limiting structure includes at least one of a pinch point; a dam formed from a dissolvable material, wherein the dissolvable material is dissolvable by the sample fluid; a hydrophilic region; and a hydrophobic region.

130. The lateral flow assay device of claim 1, wherein the diversion pad includes at least one of cellulose, glass fiber, cotton, rayon, a woven mesh, a synthetic non-woven material, and at least one superabsorbent material.

131. The lateral flow assay device of claim 26, wherein the binding component is adapted to bind at least one of a marker related to inflammation; a marker related to tissue damage; a marker related to blood vessel damage; C-Reactive Protein; soluble triggering receptor expressed on myeloid cells; a marker affected by at least one of oxygen concentration, carbon dioxide concentration, and Ph; a marker related to sepsis; a marker related to enteric bacteria; a marker related to mycobacteria; a marker related to coliform bacteria; and tuberculosis Mycobacterium tuberculosis cell wall antigen lipoarabinomannan.

132. The method of claim 43, wherein forming the loading structure includes at least one of disposing a loading pad layer between the sample pad layer and the diversion pad layer on the support; abutting the sample pad layer against the diversion pad layer at a junction; and abutting the sample pad layer against the diversion pad layer at a junction.

133. The method of claim 43, wherein forming the loading structure includes
abutting the sample pad layer against the diversion pad layer at a junction; and
at least one of disposing a loading pad layer above the junction of the diversion pad layer and sample pad layer, overlapping both the diversion pad layer and the sample pad layer; disposing a fluid impermeable barrier at the junction of the diversion pad layer and the sample pad layer; and disposing a flow limiting structure between the diversion pad and the sample pad.

134. The method of claim 133, including disposing a flow limiting structure between the diversion pad and the sample pad, wherein the flow limiting structure includes at least one of a pinch point; a dam formed from a dissolvable material, wherein the dissolvable material is dissolvable by the sample fluid; a hydrophilic region; and a hydrophobic region.

135. The method of claim 69, wherein receiving the sample fluid at the loading region of the lateral flow assay device includes at least one of receiving the sample fluid at a junction of the diversion pad and the sample pad; receiving the sample fluid at a loading pad positioned above a junction of the diversion pad and the sample pad, wherein the loading pad overlaps both the diversion pad and the sample pad; and receiving the sample fluid at a loading pad located between the diversion pad and the sample pad, wherein preferentially drawing the first portion of the sample fluid from the loading region into the diversion pad includes drawing fluid from a first side of a loading pad into the diversion pad, and wherein preferentially drawing the second portion of the sample fluid into the sample pad includes drawing fluid from a second side of the loading pad.

136. The method of claim 69, including at least one of
binding an analyte in the sample fluid to a capture component localized at a test line in the assay flow path, wherein binding of the analyte to the capture component blocks formation of a detectable signal at the test line;
solubilizing a conjugate immobilized on a conjugate pad in the assay flow path with the sample fluid, wherein the conjugate includes a binding component capable of binding specifically to an analyte in the sample fluid, and wherein the conjugate further includes a detectable component conjugated to the binding component; and
receiving the sample fluid at the loading region of the lateral flow assay device via an access port in a housing.
